(19) United States
(12) Patent Application Publication
(10) Pub. No.: US 2023/0265131 A1
(43) Pub. Date: Aug. 24, 2023

(54) AMUC-1100 POLYPEPTIDE VARIANTS FOR EFFECTING IMMUNE SIGNALLING AND/OR AFFECTING INTESTINAL BARRIER FUNCTION AND/OR MODULATING METABOLIC STATUS

(71) Applicant: Wageningen Universiteit, Wageningen (NL)

(72) Inventors: Willem Meindert De Vos, Amstedam (NL); Hanne Lore Paula Tytgat, Zurich (CH); Daniël Christianus Swarts, Bennekom (NL)

(21) Appl. No.: 18/003,974

(22) PCT Filed: Jun. 21, 2021
(86) PCT No.: PCT/EP2021/066791

§ 371 Date: Dec. 30, 2022

(30) Foreign Application Priority Data
Jul. 1, 2020 (NL) 2025968

(51) Int. Cl. C07K 14/195; C12N 15/74; A61P 3/00

(52) U.S. Cl. CPC C07K 14/195; C12N 15/74; A61P 3/00

(57) ABSTRACT

Polypeptide variants of an extracellular polypeptide of Akkermansia muciniphila are provided that are capable of modulating and/or promoting gut mucosal immune system function and/or maintaining and/or restoring metabolic status and/or increasing the physical integrity of the gut mucosal barrier in a mammal. The polypeptide variants or host cells comprising such polypeptide variants may be employed to prevent and/or treat a variety of conditions that benefit from an increased physical integrity of the gut mucosal barrier and/or an improved gut mucosal immune system function and metabolic status.

### CROSS-REFERENCE TO RELATED APPLICATIONS

This application is a national phase entry under 35 U.S.C. § 371 of International Patent Application PCT/EP2021/066791, filed Jun. 21, 2021, designating the United States of America and published as International Patent Publication WO 2022/002660 A1 on Jan. 6, 2022, which claims the benefit under Article 8 of the Patent Cooperation Treaty to Dutch Patent Application Serial No. 2025968, filed Jul. 1, 2020.

STATEMENT ACCORDING TO 37 C.F.R. § 1.821(c) or (e)—SEQUENCE LISTING SUBMITTED AS A TXT FILE

Pursuant to 37 C.F.R. § 1.821(c) or (e), a Sequence Listing ASCII text file entitled 3336-17261_ST25.txt, 26,248 bytes in size, generated Dec. 12, 2022, has been submitted, the contents of which are hereby incorporated by reference.

### TECHNICAL FIELD

The application relates to the fields of gut mucosal immune system, gut mucosal barrier, pharmaceutical, food or feed compositions comprising polypeptides and/or host cells, which are capable of modulating and/or promoting gut mucosal immune system function and/or maintaining and/or restoring and/or increasing the physical integrity of the gut mucosal barrier, and/or of maintaining, restoring or improving glucose and/or cholesterol and/or triglyceride homeostasis in a mammal (e.g., human).

### BACKGROUND

Increased permeability or hyperpermeability of the gut mucosal barrier is thought to play a role in several disorders and conditions such as bowel related diseases, autoimmune diseases, allergies, cancers, type 2 diabetes, obesity, depression, anxiety, and many others. For this reason, there has been an increased interest in understanding the role of the gut mucosal barrier dysfunction in the pathogenesis of many conditions targeting the gastrointestinal tract (GI) in mammals.

Under normal conditions, the gut mucosal barrier acts as a selective barrier permitting the absorption of nutrients, electrolytes and water and preventing the exposure to detrimental macromolecules, micro-organisms, dietary and microbial antigens (e.g., food allergens). The gut mucosal barrier is essentially composed of a layer of mucus and an underlying layer epithelial cells (referred to herein as ‘gut epithelial cells’). The gut epithelial cells are tightly linked to each other by so-called ‘tight junctions,’ which are basically ‘physical joints’ between the membranes of two gut epithelial cells. Maintenance of the gut mucosal barrier, particularly maintenance of the physical integrity of the gut epithelial cell layer (i.e., keeping the junctions between cell tight), is crucial for protection of the host against the migration of pathogenic micro-organisms, antigens, and other undesirable agents from the intestine to the blood stream.

The gut mucosal barrier is also heavily colonized by approximately 10¹²-10¹⁴ commensal microorganisms, mainly anaerobic or microaerophilic bacteria, most of which live in symbiosis with their host. These bacteria are beneficial to their host in many ways. They provide protection against pathogenic bacteria and serve a nutritional role in their host by synthesizing vitamin K and some of the components of the vitamin B complex. Further, the gut mucosal barrier has evolved a complex ‘gut mucosal immune system’ for distinguishing between commensal (i.e., beneficial bacteria) and pathogenic bacteria and other detrimental agents. The gut mucosal immune system is an integral part of the gut mucosal barrier, and comprises lymphoid tissues and specialized immune cells (i.e., lymphocytes and plasma cells), which are scattered widely throughout the gut mucosal barrier. One of the microorganisms that naturally colonizes the mucosa of healthy subjects is the mucin-degrading Akkermansia muciniphila, which has been shown to increase the intestinal barrier function (Everard et al., PNAS 110 (2013) 9066-71; Reunanen et al., Appl Environ Microbiol Mar. 20 2015), and thereby impact diseases associated with impaired gut barrier function.

Under certain circumstances, the gut mucosal barrier may be vulnerable to a wide variety of infectious organisms or agents, which are normally not able to cross the mucosal gut barrier but nevertheless manage to cross it (e.g., through gaps resulting from loose tight junctions between gut epithelia cells). Organisms or other agents that cross the gut mucosal barrier may cause diseases or other undesirable conditions (e.g., allergies) in the host. Examples of such diseases include obesity, metabolic syndrome, insulin-deficiency or insulin-resistance related disorders, type 2 diabetes, type 1 diabetes, inflammatory bowel disease (IBD), irritable bowel syndrome (IBS), glucose intolerance, abnormal lipid metabolism, atherosclerosis, hypertension, cardiac pathology, stroke, non-alcoholic fatty liver disease, alcoholic fatty liver disease, hyperglycemia, hepatic steatosis, dyslipidemias, dysfunction of the immune system associated with obesity (weight gain), allergy, asthma, autism, Parkinson's disease, multiple sclerosis, neurodegenerative diseases, depression, other diseases related to compromised barrier function, wound healing, behavioral disorders, alcohol dependence, cardiovascular diseases, high cholesterol, elevated triglycerides, atherosclerosis, sleep apnea, osteoarthritis, gallbladder disease, and cancer.

Conversely, diseases such as those mentioned above as well as other conditions such as food allergies, immaturity of the gut, e.g., due to a baby being born prematurely, exposure to radiation, chemotherapy and/or toxins, autoimmune disorders, malnutrition, sepsis, and the like, may alter the physical integrity of the gut mucosal barrier (i.e., cause loosening of the tight junctions between the gut epithelial cells), which in turn may allow undesirable micro-organism or other agents to cross the host gut mucosal barrier.

Several vaccines and/or antibodies targeted against such micro-organisms or agents have been developed over the years. However, the success of such approaches is mitigated as several micro-organisms or agents cannot be effectively targeted or eradicated with vaccines or antibodies.

Other approaches, which aim at preventing detrimental micro-organisms and other agents to cross the host's gut mucosal barrier in the first place and/or aim at preventing hyperpermeability of the gut mucosal barrier, have also been explored. For instance, compositions comprising glutamic acid have been developed to prevent and/or treat conditions associated with hyperpermeability of the gut mucosal barrier (WO 01/58283). Other substances including spermine and spermidine and precursors thereof, have also been used for the same purpose (Dorhout et al (1997). British J. Nutrition, pages 639-654). Preparations comprising arabinoxylan for promoting beneficial effects on the GI bacteria living in the vicinity of the gut mucosal barrier, have also been developed for the purpose of modulating the gut mucosal barrier (US2012/0230955).

WO2016177797 discloses a polypeptide derived from Akkermansia muciniphila i.e., the polypeptide Amuc-1100, which is capable of maintaining, restoring or increasing the physical integrity of the gut mucosal barrier and/or of maintaining, restoring or improving glucose and/or cholesterol and/or triglyceride homeostasis in a mammal and/or is capable of improving the metabolic or immune status of a mammal, inter alia by interacting with the toll-like receptor 2 (TLR2) and/or modulating TLR2 and/or the NFk-B-dependent signaling pathway, and/or promoting cytokine release (e.g., IL-6, IL-8, and IL-10) from immune cells located in the vicinity of the mucosal gut barrier of a mammal (e.g., human).

### SUMMARY OF THE DISCLOSURE

Provided are improved agents and/or compositions comprising such agents, which are suitable for maintaining and/or restoring and/or increasing the physical integrity of the gut mucosal barrier and/or preventing hyperpermeability of the gut mucosal barrier in a mammal (e.g., human), and/or for maintaining and/or restoring and/or improving glucose and/or cholesterol and/or triglyceride homeostasis in a mammal, and preferably thereby prevent or treat diseases or conditions that are associated with suboptimal permeability of the gut mucosal barrier and/or glucose and/or cholesterol and/or triglyceride homeostasis imbalance in the mammal. Alternatively or additionally, further provided are improved agents and/or compositions comprising such agents, which are suitable for modulating and/or promoting the gut mucosal immune system function in a mammal.

A distant variant of the polypeptide Amuc-1100 has been identified in Akkermansia glycaniphila that is capable of modulating and/or promoting the gut immune system function and/or maintaining and/or restoring and/or increasing the physical integrity of the gut mucosal barrier, and/or of maintaining and/or restoring and/or improving glucose and/or cholesterol and/or triglyceride homeostasis in a mammal (e.g., human). This is surprising, since previous research reports that Akkermansia glycaniphila does not have a homolog of Amuc-1100 (see Xing et al (2019); Genes & Genomics 41:1253-1264).

Without wishing to be bound by any theories, it is believed that the beneficial effects of the polypeptide of the disclosure result from the ability to interact with the TLR2 signaling pathway present at the surface of immune cells located in the vicinity of the gut mucosal barrier of a mammal. More specifically, it was found that the polypeptide as taught herein is capable of interacting with the TLR2 present at the surface of an immune cell and/or modulating and/or stimulating the TLR2-signaling pathway in an immune cell located in the vicinity of the gut mucosal barrier, so as to stimulate the secretion of cytokines (e.g., IL-6, IL-8, and IL-10) from the immune cells.

Further, it was found that the polypeptide as taught herein, is capable of modulating and/or increasing the transepithelial resistance of the gut mucosal barrier of a mammal. Since increased transepithelial resistance measurement serves as an index of decreased permeability of the gut mucosal barrier, it is believed that the polypeptides, including variants thereof, as taught herein are capable of modulating the physical integrity of the gut mucosal barrier, particularly at the level of the tight junctions between epithelial cells.

Combined together, these effects are believed to result in an improved or increased gut mucosal immune system function (e.g., greater release of cytokines at the gut mucosal barrier) as well as improved or increased physical integrity of the gut mucosal barrier, particularly at the level of the connection between gut epithelial cells (i.e., via tighter tight junctions between cells).

Additionally, it was found that treatment of HFD-fed mice with a polypeptide according to the disclosure causes a prominent decrease in body weight and fat mass gain without affecting food intake. Treatment with the polypeptide may also correct the HFD-induced hypercholesterolemia, with a significant decrease in serum HDL-cholesterol and a similar trend for LDL-cholesterol. Further, administration of the polypeptide may reduce glucose intolerance with the same or better potency as the Amuc-1100 polypeptide of Akkermansia muciniphila.

Finally, it is known that metformin stimulates the growth of Akkermansia (Lee H and Ko G, Appl Environ Microbiol. 2014 October;80(19):5935-43) and hence it is likely that Akkermansia and its extracellular peptides with similar functionality as the present polypeptide may have a similar effect as metformin on gestational diabetes and on preeclampsia (Syngelaki et al. N Engl J Med. 2016 Feb. 4; 374(5):434-43).

### Polypeptides

The disclosure teaches an isolated polypeptide characterized in that the isolated polypeptide
a) has at least 25, 26, 27, 28, 29, 30, 35, 40, 45, 50, 55, 60, 65, 70, 75, 80, 85, 90, 95, 96, 97, 98, 99, 100% sequence identity with SEQ ID NO:9 (over the entire length);
b) comprises at least 1, 2, 3, 4, 5, 6, or 7 of the following sets of amino acid residues
   i. R, S, I, S, A, and/or P (or conservative substitutions thereof) at positions that correspond to positions 1, 2, 8, 20, 23, and/or 27, respectively, in SEQ ID NO:9;
   ii. C, K, K, I, and/or T (or conservative substitutions thereof) at positions that correspond to positions 92, 93, 95, 97, and/or 100, respectively, in SEQ ID NO:9;
   iii. W, L, G, and/or F (or conservative substitutions thereof) at positions that correspond to positions 105, 106, 107, and/or 108, respectively, in SEQ ID NO:9;
   iv. F and/or E (or conservative substitutions thereof) at positions that correspond to positions 126, and/or 127, respectively, in SEQ ID NO:9;
   v. V, Y, and/or R (or conservative substitutions thereof) at positions that correspond to positions 149, 150, and/or 151, respectively, in SEQ ID NO:9;
   vi. P, E, I, F, Q, R, S, and/or V (or conservative substitutions thereof) at positions that correspond to positions 179, 181, 182, 184, 185, 188, 190, and/or 191, respectively, in SEQ ID NO:9;
   vii. P, P, P, A, A, P, G, T, A, E, A, P, Q, K, G, and/or E (or conservative substitutions thereof) at positions that correspond to positions 220, 222, 229, 230, 231, 234, 248, 258, 260, 262, 264, 172, 175, 279, 283, and/or 285, respectively, in SEQ ID NO:9.

The above-defined polypeptide can effect immune signaling and/or affect intestinal barrier function and/or affect glucose and/or cholesterol and/or triglyceride homeostasis. Preferably, the isolated polypeptide does not comprise SEQ ID NO:1 or an amino acid sequence with more than 50, 60, 70, 80, 90, 91, 92, 93, 94, 95, 96, 97, 98, 99 or 100% sequence identity with SEQ ID NO:1. The polypeptide taught herein may be capable of binding to the Toll like receptor 2 (TLR2).

In an embodiment, the above defined polypeptide is comprised in a composition, preferably further comprising a carrier, e.g., a physiologically acceptable carrier or a pharmaceutically acceptable carrier or an alimentarily acceptable carrier or a nutritionally acceptable carrier. The carrier may be any inert carrier. For instance, non-limiting examples of suitable physiologically or pharmaceutically acceptable carriers include any of well-known physiological or pharmaceutical carriers, buffers, diluents, and excipients.

In one embodiment, the polypeptides and variants thereof as taught herein are capable of stimulating the TLR2 signaling pathway in a cell, stimulating the release of cytokines from a cell (e.g., IL-6, IL-8, IL-10 and the like) and/or increasing transepithelial resistance (TER) of mammalian, e.g., human, cells, and/or improving the metabolic or immune status of a mammal, e.g., mouse or human.

As described under a), the polypeptide taught herein may also include variants of the amino acid sequence of SEQ ID NO:9, the amino acid sequences of the variants having more than 25% sequence identity with the amino acid sequence of SEQ ID NO:9. Variants of the polypeptide also include polypeptides, which have been derived, by way of one or more amino acid substitutions, deletions or insertions, from the polypeptide having the amino acid sequence of SEQ ID NO:9. Preferably, such polypeptides comprise from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions as compared to the polypeptide having the amino acid sequence of SEQ ID NO:9. As mentioned, the polypeptide may have at least 25, 30, 35, 40, 45, 50, 55, 60, 65, 70, 75, 80, 85, 90, 95, 100% sequence identity with SEQ ID NO:9, for example, at least 50% sequence identity with SEQ ID NO:9, e.g., over the entire length. The polypeptide according to the disclosure may or may not comprise a leader sequence.

In an embodiment, the polypeptide according to the disclosure comprises:
at least 5 amino acid residues as defined under i) (or conservative substitutions thereof);
at least 4 amino acid residues as defined under ii) (or conservative substitutions thereof);
at least 3 amino acid residues as defined under iii) (or conservative substitutions thereof);
at least 1 amino acid residue as defined under iv) (or conservative substitutions thereof);
at least 2 amino acid residues as defined under v) (or conservative substitutions thereof);
at least 7 amino acid residues as defined under vi) (or conservative substitutions thereof); and/or
at least 15 (or at least 12) amino acid residues as defined under vii) (or conservative substitutions thereof).

Alternatively or at the same time, the polypeptide as taught herein may comprise, specifically, the following sets of amino acid residues as defined above
i);
i) and vii);
i), ii), vi) and vii);
i), iii), iv), and vii);
i), ii), iii), iv), v), vi), vii).

Alternatively or at the same time, the polypeptide as taught herein may at least 75% sequence identity with SEQ ID NO:9, e.g., over the entire length.

In a preferred embodiment, the isolated polypeptide according to the disclosure further comprises amino acid residues S, N, E, N, (A,) P, Q, L, and/or L (or conservative substitutions thereof) at positions that correspond to positions 28, 29, 35, 37, (40,) 71, 78, 81, and/or 88, respectively, in SEQ ID NO:9. Preferably, at least 8 of these recited amino acid residues are comprised.

In yet another preferred embodiment, the isolated polypeptide according to the disclosure further comprises amino acid residues P, L, N, G, K, W, I, Y, R, I, V, L, F, and/or P (or conservative substitutions thereof) at positions that correspond to positions 116, 124, 136, 142, 148, 175, 198, 204, 212, 213, 289, 295, 298, and/or 301, respectively, in SEQ ID NO:9. Preferably, at least 13 (or at least 11) of these recited amino acid residues are comprised.

The isolated polypeptide according to the disclosure may be a natural variant of the polypeptide according to SEQ ID NO:9, e.g., a naturally occurring polypeptide with same functionality or a synthetic polypeptide with same functionality, i.e., that can effect immune signaling and/or affect intestinal barrier function and/or affect glucose and/or cholesterol and/or triglyceride homeostasis. The polypeptide may be capable of binding to the Toll like receptor 2 (TLR2).

The polypeptide as taught herein may be preceded by a N-terminal signal sequence stimulating secretion of the polypeptide from the cell. In an embodiment, the N-terminal signal sequence may be a polypeptide comprising the amino acid sequence of SEQ ID NO:3, which is the predicted naturally occurring N terminal signal sequence of the Amuc-1100 polypeptide. However, other N terminal signal sequences capable of allowing Amuc-1100 to be secreted from a cell may also be employed. For example, a truncated version or expanded version of the predicted naturally occurring N terminal signal sequence of the Amuc-1100 polypeptide may be employed, as long as such N terminal signal sequence is capable of allowing Amuc-1100 to be secreted from a cell. Alternatively, a non-naturally occurring N terminal signal sequence may be employed. The skilled person is capable of identifying N terminal signal sequences that are suitable for use in the disclosure. Thus, a polypeptide of the disclosure may comprise the amino acid sequence of SEQ ID NO:3 N terminal from its amino acid sequence.

Amino acid sequence identity may be determined by any suitable means available in the art. For instance, amino acid sequence identity may be determined by pairwise alignment using the Needleman and Wunsch algorithm and GAP default parameters as defined above. It is also understood that many methods can be used to identify, synthesize or isolate variants of the polypeptides as taught herein, such as western blot, immunohistochemistry, ELISA, amino acid synthesis, and the like.

It is also understood that any variants of the polypeptide as taught herein exert the same function and/or have the same activity as the polypeptide as taught herein. The functionality or activity of any variant may be determined by any known methods in the art, which the skilled person would consider suitable for these purposes.

### Polynucleotides

The disclosure also teaches a nucleic acid molecule, such as an isolated, synthetic or recombinant nucleic acid molecule, comprising a nucleic acid sequence that encodes the polypeptide as taught herein, for example, a nucleic acid sequence as shown in SEQ ID NO:15 or SEQ ID NO:19, or a nucleic acid sequence having at least 60, 70, 80, 90, 91, 92, 93, 94, 95, 96, 97, 98, 99, 100% sequence identity with SEQ ID NO:15 or SEQ ID NO:19.

The term “isolated nucleic acid molecule” (e.g., cDNA, genomic DNA or RNA) includes naturally occurring, artificial or synthetic nucleic acid molecules. The nucleic acid molecules may encode any of the polypeptides as taught herein. The nucleic acid molecule may be used to produce the polypeptides as taught herein. Due to the degeneracy of the genetic code various nucleic acid molecules may encode the same polypeptide (e.g., a polypeptide comprising the amino acid sequence of SEQ ID NO:9).

It is also understood that many methods can be used to identify, synthesize or isolate variants of the polynucleotide as taught herein, such as nucleic acid hybridization, PCR technology, in silico analysis and nucleic acid synthesis, and the like.

The nucleic acid molecule as taught herein may encompass a nucleic acid molecule encoding a N terminal signal sequence that is suitable for stimulating secretion of the polypeptide as taught herein from its host cell. The N terminal signal sequence encoding nucleic acid molecule may comprise the nucleic acid sequence as set forth in SEQ ID NO:4.

In an embodiment, the nucleic acid molecule as taught herein may be comprised in a chimeric gene, wherein the nucleic acid molecule is operably linked to a promoter. Thus the disclosure also relates to a chimeric gene comprising the nucleic acid molecule as taught herein.

Any promoters known in the art, and which are suitable for linkage with the nucleic acid molecules as taught herein may be used. Non-limiting examples of suitable promoters include promoters allowing constitutive or regulated expression, weak and strong expression, and the like. Any known methods in the art may be used to include the nucleic acid molecule as taught herein in a chimeric gene.

It may be advantageous to operably link the nucleic acid molecule as taught herein to a so-called ‘constitutive promoter.’

Alternatively, it may be advantageous to operably link the polynucleotides and variants thereof as taught herein to a so-called ‘inducible promoter.’ An inducible promoter may be a promoter that is physiologically (e.g., by external application of certain compounds) regulated.

The chimeric gene as taught herein may be comprised in a ‘vector’ or ‘nucleic acid construct.’ Thus the disclosure also related to vectors comprising the chimeric gene as taught herein or the nucleic acid molecule as taught herein.

In an aspect, the disclosure relates to a host cell that has been genetically modified to comprise, e.g., in its genome, a nucleic acid molecule as taught herein, a chimeric gene as taught herein or a vector as taught herein.

The genetically modified host cell as taught herein may be used to produce ex vivo and/or in vitro, the polypeptides and variants thereof as taught herein within the host cell cytoplasm or released from the cells by any means. The polypeptides as taught herein may, in particular, be expressed as a soluble or secreted molecule. The genetically modified host cells as taught herein can be any host cells suitable for transformation procedures or genetic engineering procedures. Non-limiting examples of suitable host cells include cultivable cells, such as any prokaryotic or eukaryotic cells. In an embodiment, the polypeptide according to the disclosure is expressed in bacteria, such as Escherichia coli.

In an embodiment, the host cell as taught herein may be any cell that naturally expresses the polypeptide or variant thereof taught herein. In such case, the host cell may overexpress the polypeptide or variant thereof as taught herein.

In yet an embodiment, the host cell as taught herein may be any cell that does not naturally express the polypeptide or variants thereof as taught herein.

In an embodiment, the host cell as taught herein does not belong to the species Akkermansia muciniphila or Akkermansia glycaniphila.

In another embodiment, the host cell may belong to the species Akkermansia muciniphila or Akkermansia glycaniphila and is genetically modified to comprise additional copies of the nucleic acid molecules taught herein, or to comprise a chimeric gene or vector as taught herein. Such Akkermansia muciniphila or Akkermansia glycaniphila cells may overexpress the polypeptide or a variant thereof taught herein.

The host cell as taught herein may be genetically modified using any known methods in the art. For instance, the host cells or organisms as taught herein may be genetically modified by a method comprising the step of
a) transforming the host cell with a nucleic acid molecule as taught herein, such as a nucleic acid sequence capable of encoding the polypeptides and variants thereof as taught herein;
b) culturing the host cell under conditions suitable to allow expression of the nucleic acid molecule as taught herein and/or production of the polypeptide or a variant thereof as taught herein;
c) optionally, screening for host cells capable of expressing the nucleic acid molecule as taught herein and/or producing the polypeptide or a variant thereof as taught herein.

In an embodiment, the genetically modified host cell as taught herein may belong to a species of bacteria that naturally occurs or lives in the vicinity of or within the gut mucosal barrier of a mammal. The species of bacteria are often referred to as ‘gut mucosal-associated bacteria species.’ Non-limiting examples of ‘gut mucosal-associated bacteria species’ include Akkermansia muciniphila (ATCC BAA-835), Faecalibacterium prausnitzii (A2-165), Lactobacillus rhamnosus (ATCC 53103) and Bifidobacterium breve (DSM-20213).

In certain embodiments, it may be advantageous to genetically modify a gut mucosal-associated bacteria with any of the polynucleotides and variants thereof as taught herein, for instance, to express or overexpress the polynucleotides as taught herein or to produce or overproduce the polypeptides as taught herein, directly into the vicinity of, or within the gut mucosal barrier of a mammal (e.g., human). In a preferred embodiment, the gut mucosal-associated bacteria may by any bacteria from the species Akkermansia muciniphila or Akkermansia glycaniphila. Such overproduction may be realized by genetic modification tools involving recombinant DNA technologies, genome editing such as by using tools based on CRISPR/cas-like systems, or by classical mutation selection systems.

In an embodiment, the genetically modified host cell may be any bacteria, particularly one that is not from a species of bacteria that naturally occurs or lives in the vicinity of or within the gut mucosal barrier of a mammal. Non-limiting examples of such bacteria include any beneficial isolated intestinal bacterial strains, e.g., probiotic bacteria, particularly strains selected from the genera Lactococcus, Lactobacillus, or Bifidobacterium may be used. In addition, strict anaerobic intestinal bacteria may be used such as those belonging to the genera known to occur in the human intestinal tract (Rajilic-Stojanovic & de Vos, The first 1000 cultured species of the human gastrointestinal microbiota. FEMS Microbiol Rev. 38: 996-1047).

### Methods for Producing the Polypeptide

In a further aspect, the disclosure relates to a method for producing the polypeptides, including variants, as taught herein, comprising the steps of:
(a) culturing a host cell as taught herein under conditions permitting production of the polypeptide or a variant thereof as taught herein; and
(b) optionally, isolating the polypeptide produced in step (a).

In step (a), the host cell as taught herein may be cultured according to any known culturing methods and on any known culture medium. The skilled person will be able to select a suitable host cell and will be able to establish suitable conditions allowing production of the polypeptide.

Alternatively, the polypeptide may be produced by a method comprising the steps of:
(a) culturing bacteria of the species Akkermansia muciniphila or Akkermansia glycaniphila in a suitable culture medium; and
(b) optionally, isolating the polypeptide produced in step (a).

The polypeptide produced in steps (a) of the methods above may be isolated by any known methods in the art. The skilled person will be capable of isolating the polypeptide produced from such culture medium.

Suitable culture media are, for example, taught by Derrien et al. (2004, Int. J. Syst. Evol. Microbiol. 54: 1469-76). Derrien et al. teach that A. muciniphila strain Muc^{T} was isolated and grown on a basal anaerobic medium containing hog gastric mucin as the sole carbon and nitrogen source. The authors also teach that A. muciniphila can be grown on rich media, such as Columbia Broth (CB) and Brain Heart Infusion (BHI) broth or basal medium with glucose and high concentrations of casitone and yeast-extract. Similarly, Lukovac et al. (mBio) teaches the growth of A. muciniphila in a basal medium containing glucose and fucose, as well as high amounts of casitone (2014, mBio 01438-14). Similar methods may be used for Akkermansia glycaniphila.

### Compositions

In a further aspect, the disclosure relates to a composition comprising any of the polypeptides as taught herein.

In a yet further aspect, the disclosure relates to a composition comprising a host cell as taught herein. The host cell may be present in an amount ranging from about 10⁴ to about 10¹⁵ colony forming units (CFU). For instance, an effective amount of the host cell may be an amount of about 10⁵ CFU to about 10¹⁴ CFU, preferably, about 10⁶ CFU to about 10¹³ CFU, preferably, about 10⁷ CFU to about 10¹² CFU, more preferably, about 10⁸ CFU to about 10¹² CFU. The host cell may be viable or may be dead. The effectiveness of the host cell correlates with the presence of the polypeptide as taught herein.

In an embodiment, the composition as taught herein further comprises a carrier, e.g., a physiologically acceptable carrier or a pharmaceutically acceptable carrier or an alimentarily acceptable carrier or a nutritionally acceptable carrier. The carrier may be any inert carrier. For instance, non-limiting examples of suitable physiologically or pharmaceutically acceptable carriers include any of well-known physiological or pharmaceutical carriers, buffers, diluents, and excipients. It will be appreciated that the choice for a suitable physiological or pharmaceutical carrier or alimentary carrier or nutritional carrier will depend upon the intended mode of administration of the composition as taught herein (e.g., oral) and the intended form of the composition (e.g., beverage, yogurt, powder, capsules, and the like). The skilled person knows how to select a suitable carrier, e.g., physiologically acceptable carrier or a nutritionally acceptable carrier or a pharmaceutically acceptable carrier, which is suitable for or compatible with the compositions as taught herein.

In an embodiment, the compositions as taught herein may be a nutritional, or alimentary, composition. For instance, the composition as taught herein may be a food, food supplement, feed, or a feed supplement such as a dairy product, e.g., a fermented dairy product, such as a yogurt or a yogurt drink. In this case, the composition may comprise a nutritionally acceptable or alimentarily acceptable carrier, which may be a suitable food base.

In an embodiment, the compositions as taught herein may be a pharmaceutical composition. The pharmaceutical composition may also be for use as a supplement (e.g., food supplement). The pharmaceutical composition as taught herein may comprise a pharmaceutical, nutritionally or alimentarily or physiologically-acceptable carrier, in addition to the polypeptide as taught herein and/or host cells as taught herein. The preferred form will depend on the intended mode of administration and (therapeutic) application. The carrier may be any compatible, physiologically-acceptable, non-toxic substances suitable to deliver the polypeptide as taught herein and/or host cell as taught herein to the GI tract of a mammal (e.g., human), preferably, in the vicinity of or within the gut mucosal barrier (more preferably, the colon mucosal barrier) in a mammal. For example, sterile water, or inert solids may be used as a carrier, usually complemented with a pharmaceutically acceptable adjuvant, buffering agent, dispersing agent, and the like.

The composition as taught herein may be in liquid form, e.g., a stabilized suspension of the polypeptide as taught herein or host cell as taught herein, or in solid form, e.g., a powder of lyophilized host cells as taught herein. In case the host cells as taught herein are lyophilized, a cryoprotectant such as lactose, trehalose or glycogen may be employed. For oral administration, polypeptides as taught herein or lyophilized host cells as taught herein may be administered in solid dosage forms, such as capsules, tablets, and powders, or in liquid dosage forms, such as elixirs, syrups, and suspensions. The polypeptide as taught herein or host cell as taught herein may be encapsulated in capsules such as gelatin capsules, together with inactive ingredients and powder carriers, such as e.g., glucose, lactose, sucrose, mannitol, starch, cellulose or cellulose derivatives, magnesium stearate, stearic acid, sodium saccharin, talcum, magnesium carbonate and the like.

In an embodiment, the compositions as taught herein may comprise one or more ingredients, which are suitable for promoting survival and/or viability and/or maintaining the and/or integrity of the polypeptide as taught herein and/or the host cell as taught herein during storage and/or during exposure to bile and/or during passage through the GI tract of a mammal (e.g., a human). Non-limiting examples of such ingredients include an enteric coating, and controlled release agents allowing passage through the stomach. The skilled person knows how to select suitable ingredients for ensuring that the active component (be it a polypeptide or a host cell) receives its intended destination, where it exerts its action.

In an embodiment, the compositions as taught herein may further comprise a mucosal binding agent or mucosal binding polypeptide. The term ‘mucosal binding agent’ or ‘mucosal binding polypeptide’ as used herein refers to an agent or a polypeptide that is capable of attaching itself to the gut mucosal surfaces of the gut mucosal barrier of a mammal (e.g., human).

Alternatively, use can be made of specific docking systems to attach the polypeptide as taught herein or cells producing the polypeptide or even non-producing cells that are either alive or dead. The binding can be either at the C- or N-terminus, whatever seems to be most efficient, while also the use of spacer peptides has been described. Examples include the use of LysM-based peptidoglycan binding systems (Visweswaran GR et al. 2014, Appl Microbiol Biotechnol. 98:4331-45). Moreover, a variety of mucosal binding polypeptides have been disclosed in the art. Non-limiting examples of mucosal binding polypeptide include bacterial toxin membrane binding subunits including such as the B subunit of cholera toxin, the B subunit of the E. coli heat-labile enterotoxin, Bordetella pertussis toxin subunits S2, S3, S4 and/or S5, the B fragment of Diphtheria toxin and the membrane binding subunits of Shiga toxin or Shiga-like toxins. Other suitable mucosal binding polypeptides include bacterial fimbriae proteins such as including E. coli fimbria K88, K99, 987P, F41, FAIL, CFAIII ICES1, CS2 and/or CS3, CFAIIV ICS4, CS5 and/or CS6), P fimbriae, or the like. Other non-limiting examples of fimbriae include Bordetella pertussis filamentous hemagglutinin, Vibrio cholerae toxin-coregulate pilus (TCP), Mannose-sensitive hemagglutinin (MSHA), fucose-sensitive hemagglutinin (PSHA), and the like. Still other mucosal-binding agents include viral attachment proteins including influenza and sendai virus hemagglutinins and animal lectins or lectin-like molecules including immunoglobulin molecules or fragments thereof, calcium-dependent (C-type) lectins, selectins, collectins or helix pomatis hemagglutinin, plant lectins with mucosa-binding subunits include concanavalin A, wheat-germ agglutinin, phytohemagglutinin, abrin, ricin and the like. The advantage of this delivery is that one obviates the use of a living recombinant organism.

Although not essential, it may be advantageous to add one or more mucosal binding agent or mucosal binding polypeptide to the composition as taught herein so as to target the polypeptide as taught herein or the host cell as taught herein to the gut mucosal barrier.

The compositions as taught herein may further comprise ingredients selected from the group comprising prebiotics, probiotics, carbohydrates, polypeptides, lipids, vitamins, minerals, medicinal agents, preservative agents, antibiotics, or any combination thereof.

In one embodiment, the composition as taught herein may further comprise one or more ingredients, which further enhance the nutritional value and/or the therapeutic value the compositions as taught herein. For instance, it may be advantageous to add one or more ingredients (e.g., nutritional ingredients, veterinary or medicinal agents etc.) selected from proteins, amino acids, enzymes, mineral salts, vitamins (e.g., thiamine HCl, riboflavin, pyridoxine HCl, niacin, inositol, choline chloride, calcium pantothenate, biotin, folic acid, ascorbic acid, vitamin B12, p-aminobenzoic acid, vitamin A acetate, vitamin K, vitamin D, vitamin E, and the like), sugars and complex carbohydrates (e.g., water-soluble and water-insoluble monosaccharides, disaccharides, and polysaccharides), medicinal compounds (e.g., antibiotics), antioxidants, trace element ingredients (e.g., compounds of cobalt, copper, manganese, iron, zinc, tin, nickel, chromium, molybdenum, iodine, chlorine, silicon, vanadium, selenium, calcium, magnesium, sodium and potassium and the like). The skilled person is familiar with methods and ingredients that are suitable to enhance the nutritional and/or therapeutic/medicinal value of the compositions as taught herein.

In an embodiment, the host cell may be incorporated in lyophilized form, or microencapsulated form (reviewed by, for example, Solanki et al. BioMed Res. Int. 2013, Article ID 620719), or any other form preserving the activity and/or viability of the host cell (e.g., bacterial strain).

### Methods of Treatment

In another aspect, the disclosure relates to methods for treating and/or preventing a disorder or condition selected from the group of obesity, metabolic syndrome, insulin-deficiency or insulin-resistance related disorders, type 2 diabetes, type 1 diabetes, gestational diabetes, preeclampsia, inflammatory bowel disease (IBD), irritable bowel syndrome (IBS), glucose intolerance, abnormal lipid metabolism, atherosclerosis, hypertension, cardiac pathology, stroke, non-alcoholic fatty liver disease, alcoholic fatty liver disease, hyperglycemia, hepatic steatosis, dyslipidemias, dysfunction of the immune system associated with obesity (weight gain), allergy, asthma, autism, Parkinson's disease, multiple sclerosis, neurodegenerative diseases, depression, other diseases related to compromised barrier function, wound healing, behavioral disorders, alcohol dependence, cardiovascular diseases, high cholesterol, elevated triglycerides, atherosclerosis, sleep apnea, osteoarthritis, gallbladder disease, cancer, and conditions altering the physical integrity of the gut mucosal barrier such as food allergies, immaturity of the gut, e.g., due to a baby being born prematurely, exposure to radiation, chemotherapy and/or toxins, autoimmune disorders, malnutrition, sepsis, and the like, in a mammal; methods for promoting weight loss in a mammal; methods for promoting anti-inflammatory activity in the gut of a mammal; methods for promoting gut mucosal immune system function in a mammal; methods for maintaining, restoring and/or improving glucose and/or cholesterol and/or triglyceride homeostasis; and methods for maintaining, restoring and/or increasing the physical integrity of the mucosal gut barrier in a mammal. The methods comprise the step of administering to a mammal in need thereof, an effective amount of a polypeptide as taught herein, a host cell as taught herein or a composition as taught herein.

In one embodiment, the polypeptide as taught herein, a host cell as taught herein or a composition as taught herein may be administered by any known methods of administration. For instance, the compositions as taught herein may be administered orally, intravenously, topically, enterally or parenterally. It is understood that the modes or routes of administration will depend on the case at hand (e.g., age of the subject, desired location of the effects, disease conditions and the like) as well as on the intended form of the composition (e.g., pill, liquid, powder etc.).

In a preferred embodiment, the polypeptide as taught herein, a host cell as taught herein or a composition as taught herein are administered orally.

### Uses

In a further aspect, the disclosure relates to the use of the nucleic acid molecule as taught herein, chimeric gene as taught herein and/or vectors as taught herein for producing the polypeptides as taught herein and/or for generating the host cells as taught herein. The polypeptide as taught herein and/or the host cell as taught herein may have enhanced ability to interact with the TLR2 receptor on a cell and/or may have an enhanced ability to stimulate TLR2 signaling pathway in a cell, and/or may have an enhanced ability to stimulate production of cytokines, particularly IL-1β, IL-6, IL-8, IL-10 and TNF-α, from a cell, and/or may have an enhanced ability to increase TER of mammalian, e.g., human, cells, as compared to a host cell (e.g., bacteria) not genetically modified with the polynucleotides, chimeric genes or vectors as taught herein.

In a further aspect, the disclosure relates to the polypeptide as taught herein, host cells as taught herein or composition as taught herein for use as a medicament; particularly for use in promoting gut mucosal immune system function or for maintaining, restoring and/or increasing the physical integrity of the gut mucosal barrier in a mammal; for maintaining, restoring and/or improving glucose and/or cholesterol and/or triglyceride homeostasis in a mammal; for use in preventing and/or treating a disorder or condition selected from the group comprising obesity, such as diet-induced obesity, metabolic syndrome, insulin-deficiency or insulin-resistance related disorders, type 2 diabetes, type 1 diabetes, gestational diabetes, preeclampsia, inflammatory bowel disease (IBD), irritable bowel syndrome (IBS), glucose intolerance, abnormal lipid metabolism, atherosclerosis, hypertension, cardiac pathology, stroke, non-alcoholic fatty liver disease, alcoholic fatty liver disease, hyperglycemia, hepatic steatosis, dyslipidemias, dysfunction of the immune system associated with obesity (weight gain), allergy, asthma, autism, Parkinson's disease, multiple sclerosis, neurodegenerative diseases, depression, other diseases related to compromised barrier function, wound healing, behavioral disorders, alcohol dependence, cardiovascular diseases, high cholesterol, elevated triglycerides, atherosclerosis, sleep apnea, osteoarthritis, gallbladder disease, cancer, and conditions altering the physical integrity of the gut mucosal barrier such as food allergies, immaturity of the gut, e.g., due to a baby being born prematurely, exposure to radiation, chemotherapy and/or toxins, autoimmune disorders, malnutrition, sepsis, and the like, in a mammal; for use in promoting anti-inflammatory activity in the gut of a mammal; or for use in promoting weight loss in a mammal.

In an embodiment, the mammal, e.g., human, may be of any age group (e.g., infants, adults, elderly) and of any gender (male and female). In an embodiment, the mammal may be an infant (e.g., newborns, babies, toddlers etc.), particularly an infant, which was born prematurely.

The mammal may be any mammal, for example, humans, non-human primates, rodents, cats, dogs, cow, horses, and the like. In a preferred embodiment, the mammal is a human being.

The isolated polypeptide of the disclosure may alternatively be characterized in that the polypeptide
a) has at least 25, 26, 27, 28, 29, 30, 35, 40, 45, 50, 55, 60, 65, 70, 75, 80, 85, 90, 95, 100% sequence identity with SEQ ID NO:5 (over the entire length);
b) comprises at least 1, 2, 3, 4, 5, 6, or 7 of the following sets of amino acid residues
   i. R, S, I, S, A, and/or P (or conservative substitutions thereof) at positions that correspond to positions 6, 7, 13, 22, 25, and/or 30, respectively, in SEQ ID NO:5;
   ii. C, K, K, I, and/or T (or conservative substitutions thereof) at positions that correspond to positions 88, 89, 91, 93, and/or 96, respectively, in SEQ ID NO:5;
   iii. W, L, G, and/or F (or conservative substitutions thereof) at positions that correspond to positions 101, 102, 103, and/or 10⁴, respectively, in SEQ ID NO:5;
   iv. F and/or E (or conservative substitutions thereof) at positions that correspond to positions 122, and/or 123, respectively, in SEQ ID NO:5;
   v. V, Y, and/or R (or conservative substitutions thereof) at positions that correspond to positions 145, 146, and/or 147, respectively, in SEQ ID NO:5;
   vi. P, E, I, F, Q, R, S, and/or V (or conservative substitutions thereof) at positions that correspond to positions 174, 176, 177, 179, 180, 183, 185, and/or 186, respectively, in SEQ ID NO:5;
   vii. P, P, P, A, A, P, G, T, A, E, A, P, Q, K, G, and/or E (or conservative substitutions thereof) at positions that correspond to positions 215, 217, 221, 222, 223, 226, 234, 239, 241, 243, 245, 150, 153, 257, 261, and/or 263, respectively, in SEQ ID NO:5.

The above-defined polypeptide can effect immune signaling and/or affect intestinal barrier function and/or affect glucose and/or cholesterol and/or triglyceride homeostasis. Preferably, the isolated polypeptide does not comprise SEQ ID NO:1 or an amino acid sequence with more than 50, 60, 70, 80, 90, 91, 92, 93, 94, 95, 96, 97, 98, 99 or 100% sequence identity with SEQ ID NO:1. The polypeptide may be capable of binding to the toll like receptor 2 (TLR2).

In an embodiment, the above defined polypeptide is comprised in a composition, preferably further comprising a carrier, e.g., a physiologically acceptable carrier or a pharmaceutically acceptable carrier or an alimentarily acceptable carrier or a nutritionally acceptable carrier. The carrier may be any inert carrier. For instance, non-limiting examples of suitable physiologically or pharmaceutically acceptable carriers include any of well-known physiological or pharmaceutical carriers, buffers, diluents, and excipients.

As described under a), the polypeptide may also include variants of the amino acid sequence of SEQ ID NO:5, the amino acid sequences of the variants having more than 25% sequence identity with the amino acid sequence of SEQ ID NO:5. Variants of the polypeptide also include polypeptides, which have been derived, by way of one or more amino acid substitutions, deletions or insertions, from the polypeptide having the amino acid sequence of SEQ ID NO:5. Preferably, such polypeptides comprise from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions as compared to the polypeptide having the amino acid sequence of SEQ ID NO:5. As mentioned, the polypeptide may have at least 25, 26, 27, 28, 29, 30, 35, 40, 45, 50, 55, 60, 65, 70, 75, 80, 85, 90, 95, 100% sequence identity with SEQ ID NO:5, for example, at least 50% sequence identity with SEQ ID NO:5, e.g., over the entire length. The polypeptide according to the disclosure may or may not comprise a leader sequence.

In an embodiment, the polypeptide according to the disclosure comprises:
at least 5 amino acid residues as defined under i) (or conservative substitutions thereof);
at least 4 amino acid residues as defined under ii) (or conservative substitutions thereof);
at least 3 amino acid residues as defined under iii) (or conservative substitutions thereof);
at least 1 amino acid residue as defined under iv) (or conservative substitutions thereof);
at least 2 amino acid residues as defined under v) (or conservative substitutions thereof);
at least 7 amino acid residues as defined under vi) (or conservative substitutions thereof); and/or
at least 15 (or at least 12) amino acid residues as defined under vii) (or conservative substitutions thereof).

Alternatively or at the same time, the polypeptide as taught herein may comprise, specifically, the following sets of amino acid residues as defined above
i);
i) and vii);
i), ii), vi) and vii);
i), iii), iv), and vii);
i), ii), iii), iv), v), vi), vii).

Alternatively or at the same time, the polypeptide as taught herein may at least 75% sequence identity with SEQ ID NO:5, e.g., over the entire length.

In a preferred embodiment, the isolated polypeptide according to the disclosure further comprises amino acid residues S, N, E, N, (A,) P, Q, L, and/or L (or conservative substitutions thereof) at positions that correspond to positions 34, 35, 41, 43, (46,) 67, 74, 77, and/or 84, respectively, in SEQ ID NO:5. Preferably, at least 8 of these recited amino acid residues are comprised.

In yet another preferred embodiment, the isolated polypeptide according to the disclosure further comprises amino acid residues P, L, N, G, K, W, I, Y, R, I, V, L, F, and/or P, (or conservative substitutions thereof) at positions that correspond to positions 112, 120, 132, 138, 144, 170, 193, 199, 207, 208, 297, 273, 276, and/or 279, respectively, in SEQ ID NO:5. Preferably, at least 13 (or at least 11) of these recited amino acid residues are comprised.

The isolated polypeptide according to the disclosure may be a natural variant of the polypeptide according to SEQ ID NO:5, e.g., a naturally occurring polypeptide with same functionality or a synthetic polypeptide with same functionality, i.e., that can effect immune signaling and/or affect intestinal barrier function and/or affect glucose and/or cholesterol and/or triglyceride homeostasis. The polypeptide may be capable of binding to the Toll like receptor 2 (TLR2).

The isolated polypeptide according to the disclosure may be selected from:
an isolated polypeptide that has at least 25, 30, 35, 40, 45, 50, 55, 60, 65, 70, 75, 80, 85, 90, 95, 100% sequence identity with SEQ ID NO:5 (over the entire length);
an isolated polypeptide that has at least 25, 30, 35, 40, 45, 50, 55, 60, 65, 70, 75, 80, 85, 90, 95, 100% sequence identity with SEQ ID NO:6 (over the entire length);
an isolated polypeptide that has at least 25, 30, 35, 40, 45, 50, 55, 60, 65, 70, 75, 80, 85, 90, 95, 100% sequence identity with SEQ ID NO:7 (over the entire length);
an isolated polypeptide that has at least 25, 30, 35, 40, 45, 50, 55, 60, 65, 70, 75, 80, 85, 90, 95, 100% sequence identity with SEQ ID NO:8 (over the entire length); and
an isolated polypeptide that has at least 25, 30, 35, 40, 45, 50, 55, 60, 65, 70, 75, 80, 85, 90, 95, 100% sequence identity with SEQ ID NO:9 (over the entire length),

preferably comprising the (sets) of (conserved) amino acid residues as taught herein.

### General Definitions

In the context of the disclosure, the term “polypeptide” is equivalent to the term “protein.” A polypeptide has a particular amino acid sequence. A “variant” of the polypeptide of the disclosure preferably has an amino acid sequence that has at least 25% sequence identity to a reference polypeptide. A polypeptide of the disclosure is isolated when it is no longer in its natural environment, i.e., when it is no longer present in the context of fimbriae, and/or no longer present in the context of a cell, such as an Akkermansia muciniphila or Akkermansia glycaniphila cell. A leader sequence is a region (encoded) between the promoter and the coding region and is involved in the regulation of expression. The leader sequence (or part thereof) may be translated into a leader peptide but, in contrast to signal peptides, leader peptides are at no time part of the structural proteins.

The term ‘conserved substitutions’ as used herein may refer to replacement of one or more amino acids in a polypeptide without substantial loss of functionality. It is common general knowledge that it is possible to substitute a certain amino acid by another one, without loss of activity of the polypeptide. For example, the following amino acids can typically be exchanged for one another:
Ala, Ser, Thr, Gly (small aliphatic, nonpolar or slightly polar residues)
Asp, Asn, Glu, Gln (polar, negatively charged residues and their amides)
His, Arg, Lys (polar, positively charged residues)
Met, Leu, Ile, Val (Cys) (large aliphatic, nonpolar residues)
Phe, Ty, Trp (large aromatic residues)
(refer to, for example, Schulz, G. E. et al, Principles of Protein Structure, Springer-Verlag, New York, 1979, and Creighton, T.E., Proteins: Structure and Molecular Principles, W.H. Freeman & Co., San Francisco, 1984)

Preferred “substitutions” are those that are conservative, i.e., wherein the residue is replaced by another of the same general type. In making changes, the hydropathic index of amino acids may be considered (See, e.g., Kyte et al., J. Mol. Biol. 157, 105-132 (1982)). It is known in the art that certain amino acids may be substituted by other amino acids having a similar hydropathic index or score and still result in a polypeptide having similar biological activity. In making such changes, the substitution of amino acids whose hydropathic indices are within ±2 is preferred, those that are within ±1 are more preferred, and those within ±0.5 are even more preferred. Similarly, select amino acids may be substituted by other amino acids having a similar hydrophilicity, as set forth in U.S. Pat. No. 4,554,101. In making such changes, as with the hydropathic indices, the substitution of amino acids whose hydrophilicity indices are within ±2 is preferred, those that are within ±1 are more preferred, and those within ±0.5 are even more preferred.

The term ‘sequence identity’ or ‘sequence similarity’ as used herein refers to a situation where an amino acid or a nucleic acid sequence has sequence identity or sequence similarity with another reference amino acid or nucleic acid sequence. ‘Sequence identity’ or ‘sequence similarity’ can be determined by alignment of two polypeptides or two nucleotide sequences using global or local alignment algorithms. Sequences may then be referred to as “substantially identical” or “essentially similar” when they (when optimally aligned by, for example, the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or EmbossWin version 2.10.0 (using the program “needle”). Alternatively percent similarity or identity may be determined by searching against databases, using algorithms such as FASTA, BLAST, etc. Preferably, the sequence identity refers to the sequence identity over the entire length of the sequence.

‘Transepithelial resistance’ (abbreviated as TER) is a measure of the permeability of an epithelial cell layer in vitro. Increased epithelial permeability has been linked to weakening of the tight junctions, and with decrease of TER.

The term ‘chimeric gene’ as used herein refers to any non-naturally occurring gene, i.e., a gene that is not normally found in nature in a species, in particular, a gene in which one or more parts of the nucleic acid sequence are not associated with each other in nature. For example, the promoter is not associated in nature with part or all of the transcribed region or with another regulatory region. The term ‘chimeric gene’ is understood to include expression constructs in which a heterologous promoter or transcription regulatory sequence is operably linked to one or more coding sequences, and optionally a 3′-untranslated region (3′-UTR). Alternatively, a chimeric gene may comprise a promoter, coding sequence and optionally a 3′-UTR derived from the same species, but that do not naturally occur in this combination.

The term ‘genetically modified host cell’ as used herein refers to cells that have been genetically modified, e.g., by the introduction of an exogenous nucleic acid sequence or by specific alteration of an endogenous gene sequence. Such cells may have been genetically modified by the introduction of, e.g., one or more mutations, insertions and/or deletions in the endogenous gene and/or insertion of a genetic construct (e.g., vector, or chimeric gene) in the genome. Genetically modified host cells may refer to cells in isolation or in culture. Genetically modified cells may be ‘transduced cells,’ wherein the cells have been infected with, for instance, a modified virus, e.g., a retrovirus may be used but other suitable viruses may also be contemplated such as lentiviruses. Non-viral methods may also be used, such as transfections. Genetically modified host cells may thus also be ‘stably transfected cells’ or ‘transiently transfected cells.’ Transfection refers to non-viral methods to transfer DNA (or RNA) to cells such that a gene is expressed. Transfection methods are widely known in the art, such as calcium-phosphate transfection, PEG transfection, and liposomal or lipoplex transfection of nucleic acids, and the like. Such a transfection may be transient, but may also be a stable transfection, wherein cells that have integrated the gene construct into their genome may be selected.

The term ‘effective amount’ as used herein refers to an amount necessary to achieve an effect as taught herein. For instance, an effective amount of the polypeptide or genetically engineered host cell as taught herein, is an amount that is effectively useful for modulating and/or promoting the gut mucosal immune system function and/or maintaining and/or restoring and/or increasing the physical integrity of the gut mucosal barrier (e.g., promoting formation of tighter junction between the gut epithelium cells), and/or for modulating and/or stimulating the toll-like receptor signaling pathway (i.e., TLR2 pathway) in an immune cell and/or for increasing cytokine production (e.g., IL-6, IL-8, and IL-10) in an immune cell, and/or for preventing and/or treating disorders or conditions such as obesity, metabolic syndrome, insulin-deficiency or insulin-resistance related disorders, type 2 diabetes, type 1 diabetes, inflammatory bowel disease (IBD), irritable bowel syndrome (IBS), glucose intolerance, abnormal lipid metabolism, atherosclerosis, hypertension, cardiac pathology, stroke, non-alcoholic fatty liver disease, alcoholic fatty liver disease, hyperglycemia, hepatic steatosis, dyslipidemias, dysfunction of the immune system associated with obesity (weight gain), allergy, asthma, autism, Parkinson's disease, multiple sclerosis, neurodegenerative diseases, depression, other diseases related to compromised barrier function, wound healing, behavioral disorders, alcohol dependence, cardiovascular diseases, high cholesterol, elevated triglycerides, atherosclerosis, sleep apnea, osteoarthritis, gallbladder disease, cancer, and conditions altering the physical integrity of the gut mucosal barrier such as food allergies, immaturity of the gut, e.g., due to a baby being born prematurely, exposure to radiation, chemotherapy and/or toxins, autoimmune disorders, malnutrition, sepsis, and the like.

The term ‘physiologically-acceptable carrier’ or ‘alimentarily acceptable carrier,’ ‘nutritionally acceptable carrier’ or ‘pharmaceutically-acceptable carrier’ as used herein refers to a physiologically-acceptable or alimentarily acceptable carrier or nutritionally-acceptable or pharmaceutically-acceptable carrier material, such as a liquid or solid filler, diluent, excipient, solvent or encapsulating material, involved in providing an administration form of the polypeptide or host cell of the disclosure. Each carrier must be “acceptable” in the sense of being compatible with the other ingredients of the composition and not injurious to the subject, i.e., that are suitable for consumption or nutritionally acceptable. The term ‘suitable for consumption’ or ‘nutritionally acceptable’ refers to ingredients or substances, which are generally regarded as safe for human (as well as other mammals) consumption. Non-limiting examples of materials, which can serve as physiologically-acceptable carriers or nutritionally-acceptable or pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) other non-toxic compatible substances employed in pharmaceutical formulations, and the like. Further, the terms ‘nutritionally-acceptable’ and ‘pharmaceutically acceptable’ as used herein refer to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

The term “homeostasis” refers to the property of a system in which variables are regulated so that internal conditions remain stable and relatively constant. All animals regulate their blood glucose concentration. Glucose regulation in the body is a process of keeping the body in “glucose homeostasis.” Mammals regulate their blood glucose with different hormones (e.g., insulin, glucagon, Glucagon like peptide 1, catecholamine and many others), and different nervous routes (e.g., nervous relay, gut to brain to peripheral organ axis). The human body maintains glucose levels constant most of the day, even after a 24-hour fast. Even during long periods of fasting, glucose levels are reduced only very slightly. Insulin, secreted by the beta cells of the pancreas, effectively transports glucose to the body's cells by instructing those cells to keep more of the glucose for their own use. If the glucose inside the cells is high, the cells will convert it to the insoluble glycogen to prevent the soluble glucose from interfering with cellular metabolism. Ultimately this lowers blood glucose levels, and insulin helps to prevent hyperglycemia. When insulin is deficient or cells become resistant to it, diabetes occurs. Glucagon, secreted by the alpha cells of the pancreas, encourages cells to break down stored glycogen or convert non-carbohydrate carbon sources to glucose via gluconeogenesis, thus preventing hypoglycemia. Numerous other factors and hormones are involved in the control of glucose metabolism (e.g., Glucagon like peptide 1, catecholamine and many others). Different mechanisms involving nervous routes are also contributing to this complex regulation.

“Cholesterol homeostasis” is a mechanism that contributes to the process of maintaining a balanced internal state of cholesterol within a living organism. Cholesterol, an essential biological molecule in the human body system, performs various physiological functions such as acting as a precursor for the production of bile acids, vitamin D, and steroid hormones. It also functions as a critical structural element in the cell membrane of every cell present in the body. Despite cholesterol's beneficial and necessary functions, an upset in cholesterol homeostasis can cause an increased risk of heart disease as well as upsetting other homeostatic feedback systems associated with cholesterol metabolism. The most conspicuous organ that controls cholesterol homeostasis is the liver because it not only biosynthesizes cholesterol released into the circulatory system, but breaks down potentially harmful, free-floating cholesterol from the bloodstream. HDLs are beneficial in maintaining cholesterol homeostasis because they pick up and deliver potentially dangerous cholesterol directly back to the liver where it is synthesized into harmless bile acids used by the digestive system. LDLs operate less beneficially because they tend to deposit their cholesterol in body cells and on arterial walls. It is excessive levels of LDLs that have been shown to increase risk for cardiovascular disease. In healthy subjects, cholesterol homeostasis is tightly regulated by complex feedback loops. In this case, if the healthy subject eats copious amounts of dietary cholesterol, biosynthesis in the liver is greatly reduced to keep balance. In a healthy subject who has a high baseline LDL level, either from years of poor diet habits or other genetic or medical conditions, the feedback loop and systemic coping mechanism may be overwhelmed by the same copious intake, causing dangerous homeostatic imbalance.

“Triglyceride homeostasis” is a mechanism that contributes to the process of maintaining a balanced internal state of triglycerides within a living organism. Triglyceride metabolism is of great clinical relevance. Hypertriglyceridemia denotes high (hyper-) blood or serum levels (-emia) of triglycerides, the most abundant fatty molecules. Elevated levels of triglycerides are associated with atherosclerosis, even in the absence of hypercholesterolemia (high cholesterol levels), and predispose to cardiovascular disease. High triglyceride levels also increase the risk of acute pancreatitis. Additionally, elevations and increases in TG levels over time enhance the risk of developing diabetes. It has been shown that insulin resistance is associated with high levels of triglycerides (TGs).

The term ‘about,’ as used herein indicates a range of normal tolerance in the art, for example, within 2 standard deviations of the mean. The term ‘about’ can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms ‘comprising’ or ‘to comprise’ and their conjugations, as used herein, refer to a situation wherein the terms are used in their non-limiting sense to mean that items following the word are included, but items not specifically mentioned are not excluded. It also encompasses the more limiting verb ‘to consist essentially of’ and ‘to consist of’.

Reference to an element by the indefinite article ‘a’ or ‘an’ does not exclude the possibility that more than one of the elements is present, unless the context clearly requires that there be one and only one of the elements. The indefinite article ‘a’ or ‘an’ thus usually means ‘at least one.’

### BRIEF DESCRIPTION OF THE DRAWINGS

FIG. 1 shows: A) Total body weight gain (g) (n=8-10). B) Total fat mass gain (g) measured by Time domain-Nuclear magnetic resonance (n=8-10). C) daily food intake. D) Plasma VLDL, LDL and HDL cholesterol levels (n=8-10). E) Plasma glucose (mg dl⁻¹) profile and F) mean area under the curve (AUC) measured between −30 and 120 min after glucose loading (mg.dl⁻¹·min⁻¹; n=8-10). G) Ratio of the control and insulin-stimulated p-IRβ on the loading control as measured by densitometry (n=3-5). H and I) Ratio of the control and insulin-stimulated p-Akt^{thr308} and p-Akt^{ser473} on the loading control as measured by densitometry (n=3-5).

FIG. 2 shows conversed residues in natural variants of Amuc-1100 (SEQ ID NO:1, SEQ ID NO:5, SEQ ID NO:6. SEQ ID NO:7, SEQ ID NO:8 and SEQ ID NO:9). As seen from top to bottom and from left to right, the first box indicates conserved residues that point outwards, potential role in interactions. The second, third and fourth box indicate hydrophobic residues, potentially involved in structural integrity. The fifth box indicates conserved residues that point outwards, potential role in interactions. The sixth box indicates loop.

FIG. 3 shows a sequence of Amuc-1100 (SEQ ID NO:1). Conserved residues are circled, deletions are indicated in grey.

FIG. 4—The bicistronic design used in the expression plasmids. The translation of the short peptide driven by RBS1 ensures the accessibility of RBS2, which drives the translation of the protein of interest. Linearization of RBS2 ensures that potential inhibitory secondary structures at the 5′UTR are removed, boosting translation efficiency (Mutalik et al., 2013; Nieuwkoop et al., 2019).

FIG. 5 SEAP activity (in AU) of the positive (Pam3CSK4, 1 ug/ml) and negative controls (PBS and DMEM) as well as the various Amuc 1100 variants as purified after TEV cleavage (all 50 ug/ml). For the natural variants (top), the pTH00x ID refers to the plasmid name used to purify the respective proteins.

SEQUENCE LISTING

SEQ ID NO: 1: Amino acid sequence of the Amuc-1100
polypeptide (conserved residues underlined)
IVNSKRSELDKKISIAAKEIKSANAAEITPSRSSNEELEKELNRYAKAVG
SLETAYKPFLASSALVPTTPTAFQNELKTFRDSLISSCKKKNILITDTSS
WLGFQVYSTQAPSVQAASTLGFELKAINSLVNKLAECGLSKFIKVYRPQL
PIETPANNPEESDEADQAPWTPMPLEIAFQGDRESVLKAMNAITGMQDYL
FTVNSIRIRNERMMPPPIANPAAAKPAAAQPATGAASLTPADEAAAPAAP
AIQQVIKPYMGKEQVFVQVSLNLVHFNQPKAQEPSED
SEQ ID NO: 5: Amino acid sequence of Akkermansia
municiphila protein WP_094137363.1
(pTH008, conserved residues underlined)
IVNSKRSELDKKISIAAKEIKSANAAEITPCRSSNEDLEKELNRYAKAVN
SLETAYKPFLASSALVPTTPTAFQNELKTFRDSLISSCKKKNILITDTSN
WLGFQVYSTQAPSVQAASTLGFELKAINSLVNKLTECGLSKFIKVYRPQL
PIETPANNPEESDEADQSPWTPMPLEIAFQGDRESVLNAINAITGMQDYL
FTINSIRIRNERMMPPPIANPAAAKPAADQPATGAASLTPADEAAAPAAP
AIQQVIKPYMGKEQIFVQVSLNLIHFNQPKAQEPSED
SEQ ID NO: 6: Amino acid sequence of Akkermansia
municiphila protein WP_022398192.1
(pTH009, conserved residues underlined)
IVNSKRSELDKKISVASKEIKSANAAEITPSRASNEELEKELNRYAKAVT
SLETAYKPFLASSALVPTTPTAFQNELKTFRDALIASCKKKNILITDTSS
WLGFQVYSTQAPSVQAASTLGFELKAVNSLVNKLTDCGLSKFIKVYRPQL
PIENPANNPEEDADEPNQAPWTPMPLEIAFQGNRESVLKAMNAITDSQDY
LFTVNSIRIRNERMMPPPIANPAAAKPAAAQPAAGAASLTPADEAAAPAA
PAIQQLIKPYMGKEQIFVQVSLNLVHFNQPKAQEPSED
SEQ ID NO: 7: Amino acid sequence of Akkermansia
municiphila protein WP_102725837.1
(pTH010, conserved residues underlined)
MVNSKRSELDKKISVASKEIKSANAAEITPSRTSNNELEKELNRYAKAVT
NLETAYKPFLASSALVPTTPTAFQNELKTFRDALIAACKKKNIQITDTSS
WLGFQVYSTQAPSVQAASTLGFELKAVNSLANKLTDCGLTKFIKVYRPQL
PIENPANNPEEEAEEPNQAPWSPMPLEIAFQGDRESVLKAMNAITDSQDY
LFTVNSIRIRNERMMPPPIAGPAAPKPAAAQSAAGAADLRPADEAAAQSA
APAIQQVIKPYMGKEQIFVQVSLNLVHFNQPKAQEPSED
SEQ ID NO: 8: Amino acid sequence of Akkermansia
sp. KLE1797 protein WP_067981703.1
(pTH011, conserved residues underlined)
MANSERSDLDKKIKSASQEIKSANAAAITPSHTSNKELEKELNRYAKAIG
NLETAYKPFMASSVLAPTTPTAFQNELKAFRESLIASCKEKNIQITDTSS
WLGFQLYSTQAPSVQATPTLTFEMKAINSLVNKLTDCGLTKFIKVYRSQL
PIENPARNTEDEEDSDQKAPWTGMPLEIAFQGDRGSVLKAMNAITDSQEY
LFTVNSIRIRNERMMPPPITNPAAAQPASAQPQTGAASLTPAGEAAAPAE
PPIQQIIKPYMGKEQVMVQVSLNLVHFAQPKAQEPSED
SEQ ID NO: 9: Amino acid sequence of Akkermansia
glycaniphila protein WP 067777749.1
(pTH012, conserved residues underlined)
RSASQDNIASIEEGQSTLDSDRAKRFPSNEQSLPEVNAAATRAAAIKEQI
LASTASFGQTVETATTVDGRPINGKELQDKLNTLHNKLEQLCKEKDIKLT
PEASWLGFSAFRSVTPNESDAPDLSFELSGIDHFVNTVAANGAVSITKVY
RPTVSEPADKTGKPKPAAKKNTGDWNTLPFEISFQAKRGSVGSILESIAQ
DKEYCYYITGMRIASDLTTPVPLDPFKKPAAPQPEETATAVSDIIDDGLG
GGDPLGGTPAAEPAPAPEEVRPAAQTVAKQILGNETIRVYIACELVRFNT
P

### EXAMPLES

### Example 1: Generation of Bacteria Genetically Modified to Produce Amuc-1100 Proteins

Method:

The polynucleotide encoding the mature Amuc-1100 (nucleotide sequence of SEQ ID NO:2) was cloned into E. coli TOP10 with a C-terminal His-Tag under control of the inducible T7 promoter of pET28-derivatives and introduced into E. coli BL21(DE3) for overproduction. For this purpose an ATG start codon was added to the nucleotide sequence of SEQ ID NO;2, so that the resulting polypeptide started with the amino acid sequence MIVNS. All constructs were confirmed by Sanger sequence analysis. The constructs carrying the overexpressed Amuc-1100 resulted in overproduction of soluble Amuc-1100 proteins that were purified to apparent homogeneity by Ni-column affinity chromatography and used in a concentration of 100-300 μg/ml. The purified Amuc-1100 was used to generate antibodies in rabbits essentially as described previously (Reunanen J et al. 2012, Appl Environ Microbiol 78:2337-44).

Results:

The results show that E. coli transformed with the polynucleotide (SEQ ID NO:2) was able to produce the Amuc-1100 protein in a soluble form that could be isolated easily using Ni-column chromatography as described (Tailford LE et al. 2015, Nat Commun. 6:7624). Similar results can be obtained with the polynucleotide SEQ ID NO:15 or SEQ ID NO:19.

### Example 2: Interaction and Stimulation of the TLR2 Signaling Pathway

Method:

In order to test the ability of Amuc-1100 to bind the TLR2 and other TLR receptors and subsequently stimulate the TLR2 and other TLR signaling pathways, reporter cell lines expressing TLR2 and TLR4 receptors were prepared. The ability of Amuc-1100 to bind cell lines expressing TLR2 or TLR4 and thereafter stimulate the TLR2 and/or TLR4 signaling pathway in the cells was tested in vitro by measuring the production of NF-kB from the reporter cells.

Briefly, hTLR2 and hTLR4 cell lines (Invivogen, Calif., USA) were used. Stimulation of the receptors with the corresponding ligands activates NF-κB and AP-1, which induces the production of Secreted embryonic alkaline phosphatase (SEAP), the levels of which can be measured by spectrophotometer (Spectramax). All cell lines were grown and subcultured up to 70-80% of confluency using as a maintenance medium Dulbecco's Modified Eagle Medium (DMEM) supplemented with 4.5 g/l D-glucose, 50 U/ml penicillin, 50 μg/ml streptomycin, 100 μg/m1Normocin, 2 mM L-glutamine, and 10% (v/v) of heat-inactivated Fetal Bovine Serum (FBS). For each cell line, an immune response experiment was carried out by adding 20 μl of Amuc-1100 suspensions. The reporter cells were incubated with Amuc-1100 for 20-24 h at 37° C. in a 5% CO2 incubator. Receptor ligands Pam3CSK4 (10 ng/ml for hTLR2) and LPS-EB (50 ng/ml for hTLR4) were used as positive control whereas maintenance medium without any selective antibiotics was used as negative control. SEAP secretion was detected by measuring the OD600 at 15 min, 1 h, 2 h, and 3 h after addition of 180 μL of QUANTI-Blue (Invivogen, Calif., USA) to 20 μL of induced hTLR2 and hTLR4 supernatant. Experiments were performed in triplicate.

Results:

The results show that Amuc-1100 was able to interact with TLR2. Further, the results show that Amuc-1100 exerted immune-stimulatory effects on reporter cells expressing TLR2, i.e., Amuc-1100 was capable of stimulating the release of NF-κB from reporter cells. Similar results can be obtained with the polypeptide of SEQ ID NO:5, SEQ ID NO:6, SEQ ID NO:7, SEQ ID NO:8, or SEQ ID NO:9.

### Example 3. Stimulation of Cytokine Release from Peripheral Blood Mononuclear Cells

Method:

The ability of Amuc-1100 to stimulate cytokine production or release from peripheral blood mononuclear cells (PBMCs) was tested in vitro. Briefly, peripheral blood of three healthy donors was received from the Sanquin Blood Bank, Nijmegen, The Netherlands. Peripheral blood mononuclear cells (PBMCs) were separated from the blood of healthy donors using Ficoll-Paque Plus gradient centrifugation according to the manufacturer's protocol (Amersham biosciences, Uppsala, Sweden). After centrifugation the mononuclear cells were collected, washed in Iscove's Modified Dulbecco's Medium (IMDM)+Glutamax (Invitrogen, Breda, The Netherlands) and adjusted to 0.5×10⁶ cells/ml in IMDM+Glutamax supplemented with penicillin (100 U/ml) (Invitrogen), streptomycin (100 μg/ml) (Invitrogen), and 10% heat inactivated FBS (Lonza, Basel, Switzerland). PBMCs (0.5×10⁶ cells/well) were seeded in 48-well tissue culture plates. For each donor, a negative control (medium only) was used.

The PBMCs were stimulated with A. muciniphila cells (1:10 ratio to PBMCs) either alive or heated for 10 min at 99° C.) or Amuc-1100 for 1 day and subsequently the production of cytokine IL-6, IL-8, IL-10, TNF-α, IL-1β and IL-12p70 was measured in culture supernatants using multiple analysis (Human inflammation CBA kit, Becton and Dickinson) according to the manufacturer's protocol on a FACS CantoII (Becton Dickinson) and analyzed using BD FCAP software (Becton Dickinson). The detection limits according to the manufacturer were as follows: 3.6 pg/ml IL-8, 7.2 pg/ml IL-1β, 2.5 pg/ml IL-6, 3.3 pg/ml IL-10, 3.7 pg/ml TNF-α, 1.9 pg/ml IL-12p70.

Results:

The results show that, compared to the control situation (medium only), Amuc-1100 was able to stimulate the production of cytokines, i.e., increased levels of IL-1β, IL-6, IL-8, IL-10 and TNF-α were observed. The level of cytokine induced by 4.5 μg/ml Amuc-1100 was at a similar level as that of 5 X10⁶ cells of A. muciniphila either alive or in a heat-killed form (see Table 1 below).

TABLE 1
Levels of cytokine induced by Amuc-1100 and Akkermansia
muciniphila either alive or in a heat-killed form

| Cytokine | Live | Heat-killed | Amuc-1100 |
|---|---|---|---|
| (pg/ml) | A. muciniphila | A. muciniphila | (4.5 μg/ml) |
| IL-1β | 894 ± 298 | 392 ± 71 | 504 ± 227 |
| IL-6 | 18029 ± 309 | 13477 ± 2014 | 12508 ± 2362 |
| IL-8 | 60018 ± 18229 | 54230 ± 9030 | 45432 ± 12507 |
| IL-10 | 823 ± 310 | 638 ± 118 | 526 ± 180 |
| TNF-α | 1920 ± 349 | 957 ± 568 | 1317 ± 885 |
| IL-12p70 | <2 | < 2 | <2 |

Similar results can be obtained with the polypeptide of SEQ ID NO:5, SEQ ID NO:6, SEQ ID NO:7, SEQ ID NO:8, or SEQ ID NO:9.

### Example 4: Modulation of the Transepithelial Resistance (TER)

Method:

The ability of Amuc-1100 to promote the integrity of gut epithelial cell layer was assessed by measuring the ability of Amuc-1100 to stimulate or increase TER of Caco-2 cells in vitro. Briefly, Caco-2 cells (5×10⁴ cells/insert) were seeded in Millicell cell culture inserts (3 μm pore size; Millipore) and grown for 8 days. Bacterial cells were washed once with RPMI 1640, and applied onto the inserts at OD600 nm of 0.25 (approximately 10⁸ cells) in RPMI 1640. Purified Amuc-1100 was applied onto the inserts at concentrations of 0.05, 0.5 and 5 μg/ml. The transepithelial resistance was determined with a Millicell ERS-2 TER meter (Millipore) from cell cultures at time points 0 h, and 24 h after addition of Amuc-1100.

Results:

The results showed that already 0.05 μg/ml of Amuc-1100 was able to significantly increase TER after 24 h of co-cultivation with the Caco-2 cells at a similar level of approximately 10⁸ A. muciniphila cells. Similar results can be obtained with the polypeptide of SEQ ID NO:5, SEQ ID NO:6, SEQ ID NO:7, SEQ ID NO:8, or SEQ ID NO:9.

### Example 5: Modulation of Diet-Induced Metabolic Dysfunction

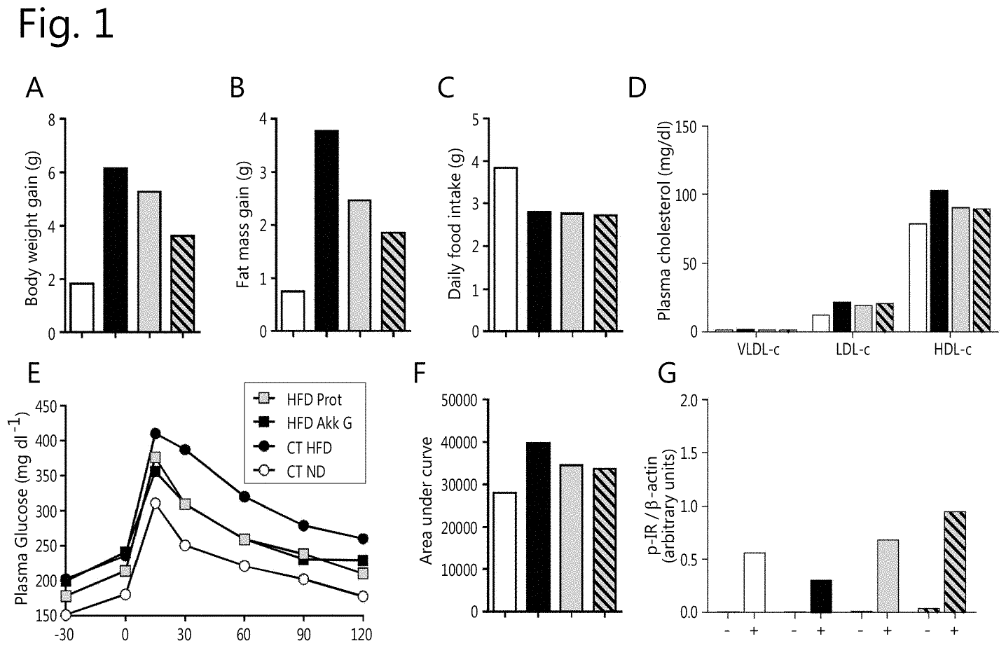

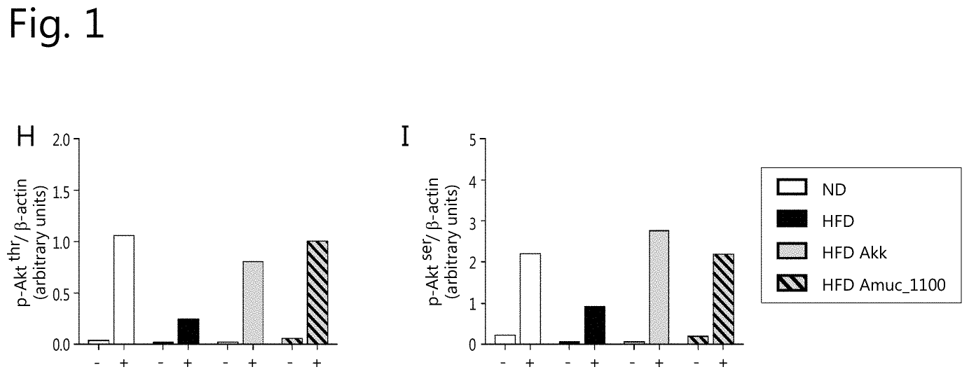

A cohort of 10-11 week-old C57BL/6J mice (n=10 per subset) was fed a control diet (ND) or an HF diet (HFD); 60% fat and 20% carbohydrates (kcal/100 g) D12492i, Research Diet, New Brunswick, N.J., USA) as previously described by Everard et al. (2013. PNAS. Vol. 110(22):9066-9071). A. muciniphila Muc^{T} was grown on a synthetic medium (containing per liter deionized water: 0.4 g KH₂PO₄, 0.669 g Na₂HPO₄·2H₂O, 0.3 g NH₄Cl, 0.3 g NaCl, 0.1 g MgCl₂·6 H₂O, 10 g Casitone, 1 mM L-threonine, 1 ml trace mineral solution, 5 mM L-fucose and 5 mM D-glucose) as described by Lucovac et al. (2014, mBio 01438-14) and concentrated, formulated in PBS containing 25% glycerol, and stored at −80° C. as described by Everard et al. supra. A subset of mice receiving HFD additionally received, daily and by oral gavage, 2×10⁸ cfu/0.15 ml A. muciniphila suspended in sterile anaerobic PBS (HFD Akk)—since this included a 10-fold dilution of the A. muciniphila, a final concentration of 2.5% glycerol was obtained. The ND and HFD groups were treated daily with an oral gavage of an equivalent volume of sterile anaerobic PBS containing 2.5% glycerol, as previously described by Everard et al., supra. A further subset of mice receiving HFD additionally received Amuc-1100 peptide delivered by daily oral gavage of 3.1 μg of the protein Amuc-1100 in an equivalent volume of sterile PBS containing 2.5% glycerol. Treatment of HFD-fed mice with Amuc-1100 caused a similar or even more prominent decrease in body weight and fat mass gain when compared to the live A. muciniphila bacterium (FIGS. 1A and B), without affecting food intake (FIG. 1C). Treatment with A. muciniphila or Amuc-1100 also corrected the HFD-induced hypercholesterolemia, with a significant decrease in serum HDL-cholesterol and a similar trend for LDL-cholesterol (FIG. 1D).

Remarkably, treatment with Amuc-1100 led to a significant decrease of serum triglycerides when compared to untreated HFD-fed mice. Moreover, Amuc-1100 treatment also reduced the adipocyte mean diameter from 38 micrometer in HFD-fed mice to 29 micrometer, a similar diameter as found in untreated mice (27 micrometer).

Interestingly, administration of Amuc-1100 reduced glucose intolerance with the same potency as the live bacterium (FIGS. 1E-F).

To further investigate glucose metabolism insulin sensitivity was investigated by injecting insulin in the portal vein. Insulin-induced phosphorylation of the insulin receptor (IR) and its downstream mediator Akt were analyzed in the liver at the threonine (Akt^{thr}) and serine (Akt^{ser}) sites (FIG. 1G). Administration of the HFD led to a decreased phosphorylation of all proteins when compared to mice fed a control chow, reaching significance in the case of Akt (FIG. 1H). Treatment with live A. muciniphila or Amuc-1100 counteracted these effects, with significantly higher levels of p-IR and p-Akt in mice treated with Amuc-1100 (FIGS. 1G-H) and significantly higher levels of p-Akt′ in mice treated with the live bacterium (FIG. 1I) when compared to the untreated HFD-fed mice. Similar results can be obtained with the polypeptide of SEQ ID NO:5, SEQ ID NO:6, SEQ ID NO:7, SEQ ID NO:8, or SEQ ID NO:9.

### Example 6: Comparative Analysis of Amuc-1100 Natural Variants and Mutants

Aim and Approach

This study aims to understand the signaling capacity of Amuc-1100 to Toll-Like Receptor 2 (TLR2) from a structure-activity perspective (Derrien et al., 2004; Plovier et al 2007). This is approached by determining the TLR2 signaling capacity of natural variants with variable sequence identity compared to Amuc-1100 protein of the type strain of Akkermansia muciniphila Amuc^{T}, as well as that of deletion mutants of Amuc-1100. All proteins, including Amuc-1100 and both natural and structural variants, were expressed without the N-terminal membrane-anchor containing signal peptide (ASP) sequence, to ensure their solubility in the cytosol of Escherichia coli, the expression host.

Natural Variants

Four proteins were identified in related A. muciniphila strains with an amino acid identity above 80% to the Amuc-1100 protein of Amuc^{T} (pTH008, SEQ ID NO:5, pTH009, SEQ ID NO:6, pTH010, SEQ ID NO:7, pTH011, SEQ ID NO:8). Additionally, a more distant variant from Akkermansia glycaniphila with only 28% sequence identity was identified (pTH012, SEQ ID NO:9).

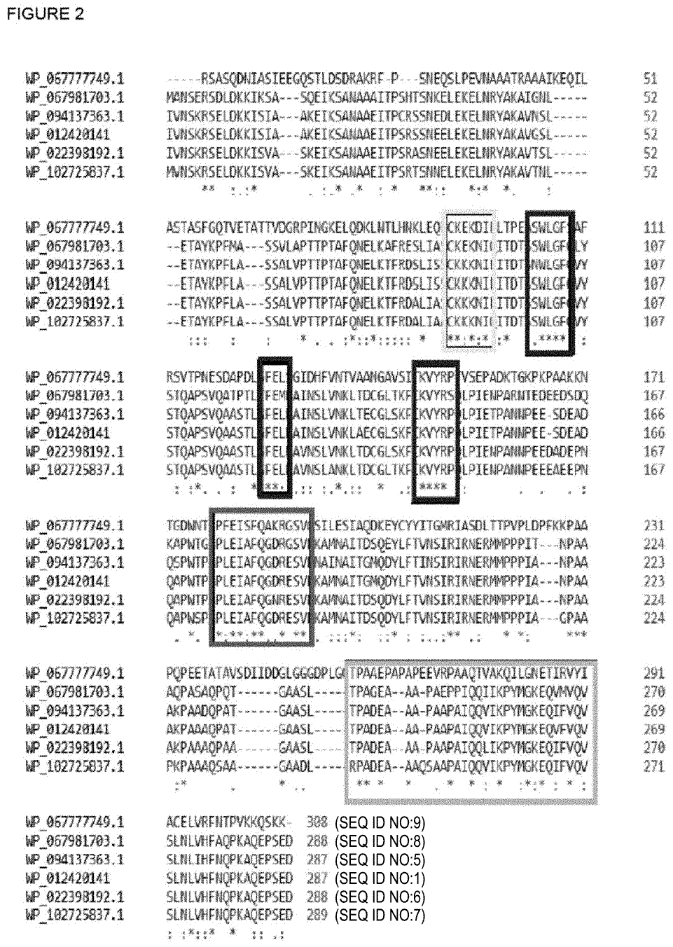

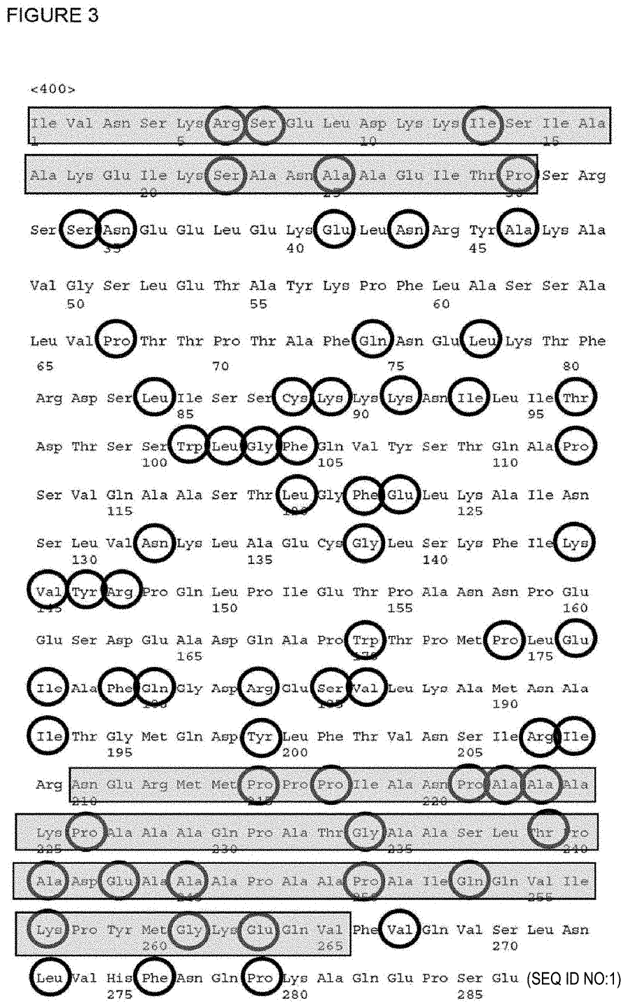

These 5 proteins are referred to as natural variants of Amuc-1100. Table 2 and FIG. 2 show conserved residues in the studied natural variants.

TABLE 2
The conserved residues in the studied natural variants (numbering
with respect to Amuc-1100, SEQ ID NO: 1).

| | | AMINO ACID | AMINO ACID |
|---|---|---|---|
| | SEQUENCE | 3-LETTER | 1-LETTER |
| | LOCATION | CODE | CODE |
| N-terminus | 6 | Arg | R |
| | 7 | Ser | S |
| | 13 | Ile | I |
| | 22 | Ser | S |
| | 25 | Ala | A |
| | 30 | Pro | P |
| | 34 | Ser | S |
| | 35 | Asn | N |
| | 41 | Glu | E |
| | 43 | Asn | N |
| | 67 | Pro | P |
| | 74 | Gln | Q |
| | 77 | Leu | L |
| | 84 | Leu | L |
| surface residues, | 88 | Cys | C |
| involved in binding | 89 | Lys | K |
| | 91 | Lys | K |
| | 93 | Ile | I |
| | 96 | Thr | T |
| hydrophobic residues | 101 | Trp | W |
| involved in structural | 102 | Leu | L |
| integrity | 103 | Gly | G |
| | 104 | Phe | F |
| | 112 | Pro | P |
| | 120 | Leu | L |
| hydrophobic residues | 122 | Phe | F |
| involved in structural | 123 | Glu | E |
| integrity | | | |
| | 132 | Asn | N |
| | 138 | Gly | G |
| | 144 | Lys | K |
| hydrophobic residues | 145 | Val | V |
| involved in structural | 146 | Tyr | Y |
| integrity | 147 | Arg | R |
| | 170 | Trp | W |
| surface residues, | 174 | Pro | P |
| involved in binding | 176 | Glu | E |
| | 177 | Ile | I |
| | 179 | Phe | F |
| | 180 | Gln | Q |
| | 183 | Arg | R |
| | 185 | Ser | S |
| | 186 | Val | V |
| | 193 | Ile | I |
| | 199 | Tyr | Y |
| | 207 | Arg | R |
| | 208 | Ile | I |
| loop | 215 | Pro | P |
| | 217 | Pro | P |
| | 221 | Pro | P |
| | 222 | Ala | A |
| | 223 | Ala | A |
| | 226 | Pro | P |
| | 234 | Gly | G |
| | 239 | Thr | T |
| | 241 | Ala | A |
| | 243 | Glu | E |
| | 245 | Ala | A |
| | 250 | Pro | P |
| | 253 | Gln | Q |
| | 257 | Lys | K |
| | 261 | Gly | G |
| | 263 | Glu | E |
| | 267 | Val | V |
| | 273 | Leu | L |
| | 276 | Phe | F |
| | 279 | Pro | P |

Gene Synthesis and Cloning

Next, the DNA coding sequences for the protein sequences of the natural variants were designed by excluding the predicted signal peptide that was detected using SignalP 5.0 (Almagro Armenteros et al., 2019) into pTN0003, ultimately resulting in pTN0005. In this plasmid (pTN0005), the exact coding sequence of Amuc-1100 of Amuc^{T} was used, as this had been shown to lead to significant overexpression in E. coli as shown previously (Plovier et al., 2017).

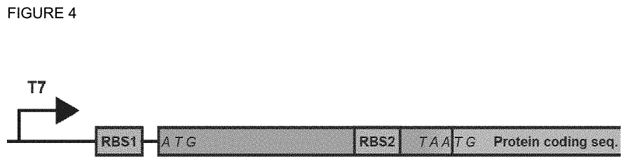

The pTN0003 vector, used as backbone for all expression constructs, contains a p15A origin, a kanamycin resistance gene, a T7 promoter, and a bicistronic design, which was followed by a terminator sequence (Mutalik et al., 2013; Nieuwkoop et al., 2019) (see FIG. 4 for an overview). An overview of the elements in the pTN0003 expression plasmid backbone is shown in Table 3.

TABLE 3
Gene elements located on the expression plasmids.
Bold or italic identify part of the sequence given.

| Sequence | | |
|---|---|---|
| Order | Description | Sequence 5′−>3′ |
| 1 | T7 promoter and bicistronic | TAATACGACTCACTATAGGGGCCCAAGTTCACTTA |
| | design | AAAAGGAGATCAACAATGAAAGCAATTTTCGTACT |
| | | GAAACATCTTAATCATGCAGGGGAGGGTTTCTA |
| | | (SEQ ID NO: 10) |
| 2 | 5′UTR TAG, containing a His | ATGCATCATCATCATCATCATCATGAAAACCTGTA |
| | tag and a TEV cleavage site | CTTCCAATCC (SEQ ID NO: 11) |
| 3 | Protein specific coding region | See Table 5 |
| 4 | 3′UTR spacer sequence and | TGCCGACTCAGTTGCTGCTTCTACTGGGCGCCCCG |
| | terminator | CTTCGGCGGGGTTTTTTT (SEQ ID NO: 12) |

For the five natural variants (pTH008, pTH009, pTH010, pTH011, pTH012), the protein sequences were reverse translated and the DNA coding sequence was optimized for expression in E. coli by using Benchling's Codon Optimization Tool (based on DNAChisel) (Benchling, 2018). For these natural variants and for the Amuc-1100 sequence of pTN0005, the DNA was ordered as gBlocks (Integrated DNA technologies). The DNA fragments were subsequently cloned into a PCR-amplified linear pTN0003 vector via Gibson assembly (primers in Table 4). The protein coding sequences ultimately introduced for each variant in the expression plasmids are provided in Table 5.

TABLE 4
Overview of the primers used for creation of the pTN0003 backbone.

| Primer | Sequence 5′−>3′ | Used for |
|---|---|---|
| pTN0003 backbone fwd | tgccgactcagttgctgc | Expression plasmid backbone fwd |
| | (SEQ ID NO: 13) | |
| pTN0003 backbone rvs | ggattggaagtacaggttttcatgatg | Expression plasmid backbone rvs |
| | (SEQ ID NO: 14) | |

TABLE 5
Amuc-1100 natural variant coding sequences as used in the expression plasmids.

| Description | Sequence 5′−>3′ |
|---|---|
| ACD04926 ΔSP Native | ATCGTCAATTCCAAACGCAGTGAACTGGACAAAAAAATCAGCATCGCCGCCAA |
| Amuc_1100 form AmucT | GGAAATCAAGTCCGCCAATGCTGCGGAAATCACTCCGAGCCGATCATCCAACG |
| (pTN0005, SEQ ID NO: 2) | AAGAGCTGGAAAAAGAACTGAACCGCTATGCCAAGGCCGTGGGCAGCCTGGAA |
| | ACGGCCTACAAGCCCTTCCTTGCCTCCTCCGCGCTGGTCCCCACCACGCCCAC |
| | GGCATTCCAGAATGAACTGAAAACATTCAGGGATTCCCTGATCTCCTCCTGCA |
| | AGAAAAAGAACATTCTCATAACGGACACATCCTCCTGGCTCGGTTTCCAGGTT |
| | TACAGCACCCAGGCTCCCTCTGTTCAGGCGGCCTCCACGCTGGGTTTTGAATT |
| | GAAAGCCATCAACAGCCTGGTCAACAAACTGGCGGAATGCGGCCTGTCCAAAT |
| | TCATCAAGGTGTACCGCCCCCAGCTCCCCATTGAAACCCCGGCGAACAATCCG |
| | GAAGAATCGGACGAAGCCGACCAGGCCCCATGGACTCCCATGCCTCTGGAAAT |
| | AGCCTTCCAGGGCGACCGGGAAAGTGTATTGAAAGCCATGAACGCCATAACCG |
| | GCATGCAGGACTATCTGTTCACGGTCAACTCCATCCGTATCCGCAACGAACGG |
| | ATGATGCCCCCTCCCATCGCCAATCCGGCAGCCGCCAAACCTGCCGCGGCCCA |
| | ACCCGCCACGGGTGCGGCTTCCCTGACTCCGGCGGATGAGGCGGCTGCACCTG |
| | CAGCCCCGGCCATCCAGCAAGTCATCAAGCCTTACATGGGCAAGGAGCAGGTC |
| | TTTGTCCAGGTCTCCCTGAATCTGGTCCACTTCAACCAGCCCAAGGCTCAGGA |
| | ACCGTCTGAAGATTAA |
| WP_094137363.1 ΔSP | ATCGTCAATAGTAAACGCTCTGAGCTGGATAAGAAGATCTCTATCGCCGCTAA |
| (pTH008, SEQ ID NO: 15) | GGAGATCAAGTCGGCTAATGCCGCCGAGATCACCCCCTGTCGCTCTAGTAATG |
| | AGGATCTTGAGAAAGAGCTGAATCGCTATGCCAAGGCGGTCAATAGTCTGGAG |
| | ACCGCCTATAAACCTTTTCTGGCCTCGAGTGCCCTTGTTCCCACCACCCCTAC |
| | CGCCTTTCAGAATGAACTGAAAACATTCAGAGACAGCCTAATAAGCAGCTGCA |
| | AAAAAAAAAACATACTAATAACAGACACAAGCAACTGGCTAGGATTCCAAGTA |
| | TACAGCACACAAGCACCAAGCGTACAAGCAGCAAGCACACTAGGATTCGAACT |
| | AAAAGCAATAAACAGCCTAGTAAACAAACTAACAGAATGCGGACTAAGCAAAT |
| | TCATAAAAGTATACAGACCACAACTACCAATAGAAACACCAGCAAACAACCCA |
| | GAAGAAAGCGACGAAGCAGACCAAAGCCCATGGACACCAATGCCACTAGAAAT |
| | AGCATTCCAAGGAGACAGAGAAAGCGTACTAAACGCAATAAACGCAATAACAG |
| | GAATGCAAGACTACCTATTCACAATAAACAGCATAAGAATAAGAAACGAAAGA |
| | ATGATGCCACCACCAATAGCAAACCCAGCAGCAGCAAAACCAGCAGCAGACCA |
| | ACCAGCAACAGGAGCAGCAAGCCTAACACCAGCAGACGAAGCAGCAGCACCAG |
| | CAGCACCAGCAATACAACAAGTAATAAAACCATACATGGGAAAAGAACAAATA |
| | TTCGTACAAGTAAGCCTAAACCTAATACACTTCAACCAACCAAAAGCACAAGA |
| | ACCAAGCGAAGACTAA |
| WP 022398192.1 ΔSP | ATCGTCAATTCGAAGCGCTCAGAGCTGGATAAGAAGATCTCCGTTGCCAGTAA |
| (pTH009, SEQ ID NO: 16) | AGAGATCAAGAGTGCCAATGCCGCTGAGATCACCCCCTCGCGCGCCTCTAATG |
| | AGGAACTGGAGAAAGAACTTAATCGCTATGCCAAAGCCGTTACCAGTCTGGAG |
| | ACCGCCTATAAGCCCTTTCTGGCCTCTTCTGCCCTGGTCCCCACCACTCCTAC |
| | CGCCTTTCAGAATGAGCTGAAAACATTCAGAGACGCACTAATAGCAAGCTGCA |
| | AAAAAAAAAACATACTAATAACAGACACAAGCAGCTGGCTAGGATTCCAAGTA |
| | TACAGCACACAAGCACCAAGCGTACAAGCAGCAAGCACACTAGGATTCGAACT |
| | AAAAGCAGTAAACAGCCTAGTAAACAAACTAACAGACTGCGGACTAAGCAAAT |
| | TCATAAAAGTATACAGACCACAACTACCAATAGAAAACCCAGCAAACAACCCA |
| | GAAGAAGACGCAGACGAACCAAACCAAGCACCATGGACACCAATGCCACTAGA |
| | AATAGCATTCCAAGGAAACAGAGAAAGCGTACTAAAAGCAATGAACGCAATAA |
| | CAGACAGCCAAGACTACCTATTCACAGTAAACAGCATAAGAATAAGAAACGAA |
| | AGAATGATGCCACCACCAATAGCAAACCCAGCAGCAGCAAAACCAGCAGCAGC |
| | ACAACCAGCAGCAGGAGCAGCAAGCCTAACACCAGCAGACGAAGCAGCAGCAC |
| | CAGCAGCACCAGCAATACAACAACTAATAAAACCATACATGGGAAAAGAACAA |
| | ATATTCGTACAAGTAAGCCTAAACCTAGTACACTTCAACCAACCAAAAGCACA |
| | AGAACCAAGCGAAGACTAA |
| WP_102725837.1 ΔSP | ATGGTCAATTCGAAACGCAGTGAGCTGGATAAAAAGATCTCCGTCGCCTCTAA |
| (pTH010, SEQ ID NO: 17) | AGAGATCAAGAGTGCCAATGCCGCCGAGATCACCCCTAGTCGCACCTCTAATA |
| | ATGAGCTGGAGAAAGAGCTGAATCGCTATGCCAAGGCCGTGACCAATCTGGAA |
| | ACCGCCTATAAGCCCTTTCTGGCCTCTTCGGCCCTGGTTCCTACCACCCCCAC |
| | CGCCTTTCAGAATGAGCTGAAAACATTCAGAGACGCACTAATAGCAGCATGCA |
| | AAAAAAAAAACATACAAATAACAGACACAAGCAGCTGGCTAGGATTCCAAGTA |
| | TACAGCACACAAGCACCAAGCGTACAAGCAGCAAGCACACTAGGATTCGAACT |
| | AAAAGCAGTAAACAGCCTAGCAAACAAACTAACAGACTGCGGACTAACAAAAT |
| | TCATAAAAGTATACAGACCACAACTACCAATAGAAAACCCAGCAAACAACCCA |
| | GAAGAAGAAGCAGAAGAACCAAACCAAGCACCATGGAGCCCAATGCCACTAGA |
| | AATAGCATTCCAAGGAGACAGAGAAAGCGTACTAAAAGCAATGAACGCAATAA |
| | CAGACAGCCAAGACTACCTATTCACAGTAAACAGCATAAGAATAAGAAACGAA |
| | AGAATGATGCCACCACCAATAGCAGGACCAGCAGCACCAAAACCAGCAGCAGC |
| | ACAAAGCGCAGCAGGAGCAGCAGACCTAAGACCAGCAGACGAAGCAGCAGCAC |
| | AAAGCGCAGCACCAGCAATACAACAAGTAATAAAACCATACATGGGAAAAGAA |
| | CAAATATTCGTACAAGTAAGCCTAAACCTAGTACACTTCAACCAACCAAAAGC |
| | ACAAGAACCAAGCGAAGACTAA |
| WP 067981703.1 ΔSP | ATGGCTAATTCGGAGCGCAGTGATCTGGATAAGAAAATCAAGTCTGCTAGTCA |
| (pTH011, SEQ ID NO: 18) | GGAGATCAAGAGTGCCAATGCCGCCGCCATCACCCCCTCGCATACCTCGAATA |
| | AAGAGCTGGAGAAAGAGCTGAATCGCTATGCTAAGGCTATCGGCAATCTTGAG |
| | ACCGCTTATAAGCCCTTTATGGCCTCTTCGGTTCTGGCTCCCACCACCCCTAC |
| | CGCCTTTCAGAATGAGCTGAAAGCATTCAGAGAAAGCCTAATAGCAAGCTGCA |
| | AAGAAAAAAACATACAAATAACAGACACAAGCAGCTGGCTAGGATTCCAACTA |
| | TACAGCACACAAGCACCAAGCGTACAAGCAACACCAACACTAACATTCGAAAT |
| | GAAAGCAATAAACAGCCTAGTAAACAAACTAACAGACTGCGGACTAACAAAAT |
| | TCATAAAAGTATACAGAAGCCAACTACCAATAGAAAACCCAGCAAGAAACACA |
| | GAAGACGAAGAAGACAGCGACCAAAAAGCACCATGGACAGGAATGCCACTAGA |
| | AATAGCATTCCAAGGAGACAGAGGAAGCGTACTAAAAGCAATGAACGCAATAA |
| | CAGACAGCCAAGAATACCTATTCACAGTAAACAGCATAAGAATAAGAAACGAA |
| | AGAATGATGCCACCACCAATAACAAACCCAGCAGCAGCACAACCAGCAAGCGC |
| | ACAACCACAAACAGGAGCAGCAAGCCTAACACCAGCAGGAGAAGCAGCAGCAC |
| | CAGCAGAACCACCAATACAACAAATAATAAAACCATACATGGGAAAAGAACAA |
| | GTAATGGTACAAGTAAGCCTAAACCTAGTACACTTCGCACAACCAAAAGCACA |
| | AGAACCAAGCGAAGACTAA |
| WP_067777749.1 ΔSP | CGTAGTGCTAGTCAGGATAATATCGCCTCGATCGAGGAGGGCCAGTCTACCCT |
| (pTH012, SEQ ID NO: 20) | GGATAGTGATCGCGCCAAGCGCTTTCCCTCGAATGAGCAGTCGCTGCCCGAGG |
| | TCAATGCCGCCGCTACCCGCGCTGCCGCCATCAAGGAGCAGATCCTTGCCAGT |
| | ACCGCTTCCTTTGGCCAGACCGTCGAGACCGCCACCACCGTCGATGGCCGGCC |
| | CATCAATGGCAAGGAGCTGCAGGATAAGCTGAATACCCTGCACAACAAACTAG |
| | AACAACTATGCAAAGAAAAAGACATAAAACTAACACCAGAAGCAAGCTGGCTA |
| | GGATTCAGCGCATTCAGAAGCGTAACACCAAACGAAAGCGACGCACCAGACCT |
| | AAGCTTCGAACTAAGCGGAATAGACCACTTCGTAAACACAGTAGCAGCAAACG |
| | GAGCAGTAAGCATAACAAAAGTATACAGACCAACAGTAAGCGAACCAGCAGAC |
| | AAAACAGGAAAACCAAAACCAGCAGCAAAAAAAAACACAGGAGACTGGAACAC |
| | ACTACCATTCGAAATAAGCTTCCAAGCAAAAAGAGGAAGCGTAGGAAGCATAC |
| | TAGAAAGCATAGCACAAGACAAAGAATACTGCTACTACATAACAGGAATGAGA |
| | ATAGCAAGCGACCTAACAACACCAGTACCACTAGACCCATTCAAAAAACCAGC |
| | AGCACCACAACCAGAAGAAACAGCAACAGCAGTAAGCGACATAATAGACGACG |
| | GACTAGGAGGAGGAGACCCACTAGGAGGAACACCAGCAGCAGAACCAGCACCA |
| | GCACCAGAAGAAGTAAGACCAGCAGCACAAACAGTAGCAAAACAAATACTAGG |
| | AAACGAAACAATAAGAGTATACATAGCATGCGAACTAGTAAGATTCAACACAC |
| | CAGTAAAAAAACAAAGCAAAAAATAA |

All expression plasmids were transformed into BL21(DE3) competent E. coli cells (New England Biolabs). After cloning, the expression constructs were sequence-verified.

Protein Expression and Purification

Protein Expression

Strains harboring an expression plasmid were precultured in LB medium supplemented with kanamycin (50 μg/mL). Erlenmeyer's (5 L) containing 1.5 L LB medium supplemented with Kanamycin (50 μg/mL) were inoculated with 10 mL of preculture and incubated at 37° C., 120 rpm, until the cultures reached an OD600 of 0.6-0.8. Prior to induction, flasks were put on ice for 30 min. After induction with 0.4 mM IPTG (final concentration), cultures were further incubated for 18 hours at 20° C. and 120 rpm. Cells were harvested by centrifugation and the pellet was washed with 25 mL wash buffer (50 mM NaH₂PO₄, 300 mM NaCl, 20 mM imidazole, pH 8.0). The cell pellets were stored at −80° C.

Protein Purification

Cell pellets were allowed to thaw in 25 mL wash buffer supplemented with a protease inhibitor tablet (Roche cOmplete™). The resuspended cells were sonicated (Bandelin Sonopuls, VS 70/T probe, 25% intensity, 1 second on 2 seconds off for a total time of 10 minutes, on ice). Lysed cells were centrifuged (15 min, 30 000×g, 4° C.) and filtered (0.45 μm) to remove cell debris.

Proteins were further purified exploiting their N-terminal His-tag on a 5 mL HisTrap HP column (GE Healthcare) using an Akta FPLC system. The protein was eluted in 50 mM NaH₂PO₄, 300 mM NaCl, 500 mM imidazole, pH 8.0. The His-tag was cleaved off using 0.7 mg His-tagged TEV protease during overnight dialysis (14 k MWCO) at 4° C. against wash buffer in a 1:500 ratio. In order to remove the TEV protease from the Amuc-1100 proteins, these were run a second time over the HisTrap column. This time, the flowthrough, containing the protein of interest was collected, whilst the His-tagged TEV protease remained bound to the HisTrap column.

In Vitro Culture and Stimulation of Human HEK-Blue hTLR2 Cell Lines

HEK-Blue hTLR2 cells (Invivogen, Calif., USA) were used to screen for TLR2 activation. In this cell line, stimulation of TLR2 and subsequent activation of NF-κB and AP-1 induces the production of secreted embryonic alkaline phosphatase (SEAP), which can be quantified spectrophotometrically.

The cell line was grown and subcultured up to 70-80% of confluency in a maintenance medium of Dulbecco's Modified Eagle Medium (DMEM) supplemented with GlutaMAX™, 4.5 g/L D-glucose, 100 U/mL penicillin, 100 μg/mL streptomycin, 100 μg/mL normocin, 10% (v/v) of heat-inactivated FBS and HEK-Blue™ Selection (Invivogen). Cells were maximally maintained until passage 25. TLR2 activation was tested by seeding HEK-blue cells in flat-bottom 96-well plates in maintenance medium without HEK-Blue™ Selection and stimulating them 24 h later by addition of 20 μL of the protein of interest (with a concentration of a 50 ug/mL) in triplo. The 96-well plates were incubated for 20-24 h at 37° C. in a 5% CO2 incubator. The receptor ligand Pam3CSK4 was used as positive control whereas PBS (dilution reagent of the proteins of interest) was used as a negative control. Secreted Embryonic Alkaline Phosphatase (SEAP) activity detected by measuring the absorbance at 600 nm (Synergy™ Mx, BioTek Instruments, Inc., VT, USA) at 1 h after addition of 20 μL of induced HEK-Blue hTLR2 supernatant to 180 μL of QUANTI-Blue (Invivogen) and expressed as arbitrary units (AU).

Results and Conclusions

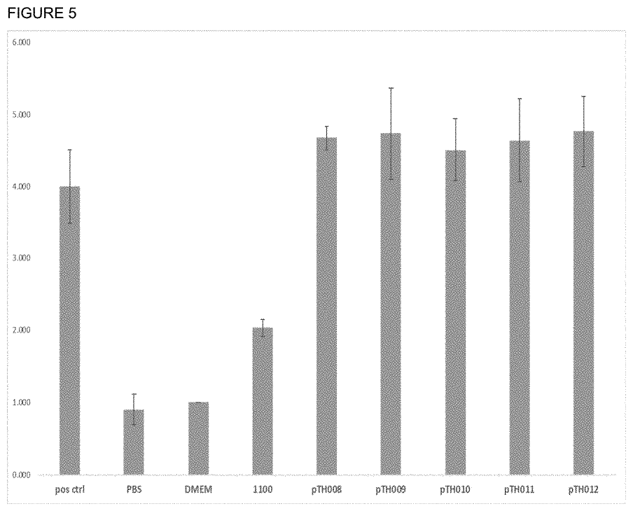

The capacity to activate TLR2 of the purified Amuc-1100 proteins from Amuc^{T} as well as the natural variants was determined as described above. The results are shown in FIG. 5.

The activity on TLR2 cells of the positive control of 1 μg/ml Pam3CSK4 amounted to approximately 4.0 AU while that of the negative controls PBS and DMEM was lower than 1.0 AU. The Amuc 1100 protein from Amuc^{T} (1100) showed a significant activity on TLR2 cells above background of approximately 2.0 AU.

As can be seen in FIG. 5, all natural variants tested here are capable of activating the TLR2 receptor. The TLR2 activation capacity of the Amuc-1100 natural variants (FIG. 5), was surprisingly higher than that of the positive control and amounted to approximately 4.5 AU.

The results of 3D modeling (data not shown) indicated that deleting the respective regions may reduce the ability of the protein to interact with the TLR2 receptor. These results point toward the importance of dimerization and the presence of the long, unordered loop of Amuc-1100 as structural features improving TLR-signaling activity.

Accordingly, the relationship between deletion of specific conserved regions and effect thereof on the capacity to activate TLR2 is assessed in Table 6:

TABLE 6
Assessment of relationship between deletion of specific conserved regions and negative
effect thereof on capacity to activate TLR2

| | | | Effect deletion of |
|---|---|---|---|
| | Numbering of amino acids | Numbering of amino acids | respective conserved |
| | with respect to SEQ ID | with respect to SEQ ID | region on capacity to |
| Conserved region | NO: 9 | NO: 5 | activate TLR2 |
| N-terminus R, S, I, S, A, | 1, 2, 8, 20, 23, 27 | 6, 7, 13, 22, 25, 30 | Very high |
| and P | | | |
| Loop P, P, P, A, A, P, G, | 220, 222, 229, 230, 231, | 215, 217, 221, 222, 223, | Very high |
| T, A, E, A, P, Q, K, G, and | 234, 248, 258, 260, 262, | 226, 234, 239, 241, 243, | |
| E | 264, 172, 175, 279, 283, | 245, 150, 153, 257, 261, | |
| | 285 | 263 | |
| Surface residues involved | 92, 93, 95, 97, 100 | 88, 89, 91, 93, 96 | High |
| in binding C, K, K, I, and | | | |
| T | | | |
| Surface residues involved | 179, 181, 182, 184, 185, | 174, 176, 177, 179, 180, | High |
| in binding P, E, I, F, Q, R, | 188, 190, 191 | 183, 185, 186 | |
| S, and V | | | |
| Hydrophobic residues | 105, 106, 107, 108 | 101, 102, 103, 104 | Medium |
| involved in structural | | | |
| integrity W, L, G, and F | | | |
| Hydrophobic residues | 126, 127 | 122, 123 | Medium |
| involved in structural | | | |
| integrity F and E | | | |
| Hydrophobic residues | 149, 150, 151 | 145, 146, 147 | Medium |
| involved in structural | | | |
| integrity V, Y, and R | | | |
| Other conserved residues | 28, 29, 35, 37, 71, 78, 81, | 34, 35, 41, 43, 67, 74, 77, | Low |
| S, N, E, N, P, Q, L, L | 88 | 84 | |
| Other conserved residues | 116, 124, 136, 142, 148, | 112, 120, 132, 138, 144, | Low |
| P, L, N, G, K, W, I, Y, R, | 175, 198, 204, 212, 213, | 170, 193, 199, 207, 208, | |
| I, V, L, F, P | 289, 295, 298, 301 | 297, 273, 276, 279 | |

In particular, the N-terminus with beta strand for dimerization and the presence of the long, unordered loop are important for (improved) TLR-signaling activity.

### REFERENCES

Almagro Armenteros, J. J., Tsirigos, K. D., Sønderby, C. K., Petersen, T. N., Winther, O., Brunak, S., . . . Nielsen, H. (2019). SignalP 5.0 improves signal peptide predictions using deep neural networks. Nature Biotechnology. doi.org/10.1038/s41587-019-0036-z
Benchling (2018). Benchling for Academics Benchling. Retrieved Sep. 13, 2019, from www.benchling.com
Derrien, M. E. E. Vaughan, C. M. Plugge & W. M. de Vos (2004) Akkermansia muciniphila gen. nov., sp. nov., a human intestinal mucin-degrading bacterium. Int. J. Syst. Evol. Microbiol. 54: 1469-76.
Mutalik, V. K., Guimaraes, J. C., Cambray, G., Lam, C., Christoffersen, M. J., Mai, Q. A., . . . Endy, D. (2013). Precise and reliable gene expression via standard transcription and translation initiation elements. Nature Methods, 10(4), 354-360. doi.org/10.1038/nmeth.2404
Nieuwkoop, T., Claassens, N. J., & van der Oost, J. (2019). Improved protein production and codon optimization analyses in Escherichia coli by bicistronic design. Microbial Biotechnology, 12(1), 173-179. doi.org/10.1111/1751-7915.13332.
Plovier, H., Everard, A., Druart, C., Depommier, C., Van Hul, M., Geurts, L., Cani, P.D. (2017). A purified membrane protein from Akkermansia muciniphila or the pasteurized bacterium improves metabolism in obese and diabetic mice. Nature Medicine, 23(1). doi.org/10.1038/nm.4236

1.-18. (canceled)

19. A composition comprising an isolated polypeptide and a pharmaceutically or alimentary acceptable carrier, wherein the isolated polypeptide
a) has at least 30% sequence identity with SEQ ID NO:9; and
b) comprises at least one of the following sets of amino acid residues
i. R, S, I, S, A, and P or conservative substitutions thereof at positions that correspond to positions 1, 2, 8, 20, 23, and 27, respectively, in SEQ ID NO:9;
ii. C, K, K, I, and T or conservative substitutions thereof at positions that correspond to positions 92, 93, 95, 97, and 100, respectively, in SEQ ID NO:9;
iii. W, L, G, and F or conservative substitutions thereof at positions that correspond to positions 10⁵, 10⁶, 10⁷, and 10⁸, respectively, in SEQ ID NO:9;
iv. F and E or conservative substitutions thereof at positions that correspond to positions 126 and 127, respectively, in SEQ ID NO:9;
v. V, Y, and R or conservative substitutions thereof at positions that correspond to positions 149, 150, and 151, respectively, in SEQ ID NO:9;
vi. P, E, I, F, Q, R, S, and V or conservative substitutions thereof at positions that correspond to positions 179, 181, 182, 184, 185, 188, 190, and 191, respectively, in SEQ ID NO:9; and
vii. P, P, P, A, A, P, G, T, A, E, A, P, Q, K, G, and E or conservative substitutions thereof at positions that correspond to positions 220, 222, 229, 230, 231, 234, 248, 258, 260, 262, 264, 172, 175, 279, 283, and 285, respectively, in SEQ ID NO:9,
wherein the polypeptide affects immune signaling, affects intestinal barrier function, affects glucose homeostasis, cholesterol homeostasis, and/or triglyceride homeostasis.

20. The composition of claim 19, wherein the isolated polypeptide comprises all of the sets i through vii of amino acid residues.

21. The composition of claim 19, wherein the isolated polypeptide further comprises amino acid residues S, N, E, N, P, Q, L, L or conservative substitutions thereof at positions that correspond to positions 28, 29, 35, 37, 71, 78, 81, and 88, respectively, in SEQ ID NO:9.

22. The composition of claim 19, wherein the isolated polypeptide further comprises amino acid residues P, L, N, G, K, W, I, Y, R, I, V, L, F, P, or conservative substitutions thereof at positions that correspond to positions 116, 124, 136, 142, 148, 175, 198, 204, 212, 213, 289, 295, 298, and 301, respectively, in SEQ ID NO:9.

23. The composition of claim 19, wherein the isolated polypeptide is a natural variant of SEQ ID NO:9.

24. The composition of claim 19, wherein the isolated polypeptide is a synthetic variant of SEQ ID NO:9.

25. The composition of claim 19, which is a nutritional composition or a pharmaceutical composition.

26. A genetically modified host cell comprising a nucleic acid molecule comprising a nucleic acid sequence encoding a polypeptide
a) having at least 30% sequence identity with SEQ ID NO:9; and
b) comprising at least one of the following sets of amino acid residues
i. R, S, I, S, A, and P or conservative substitutions thereof at positions that correspond to positions 1, 2, 8, 20, 23, and 27, respectively, in SEQ ID NO:9;
ii. C, K, K, I, and T or conservative substitutions thereof at positions that correspond to positions 92, 93, 95, 97, and 100, respectively, in SEQ ID NO:9;
iii. W, L, G, and F or conservative substitutions thereof at positions that correspond to positions 105, 106, 107, and 108, respectively, in SEQ ID NO:9;
iv. F and E or conservative substitutions thereof at positions that correspond to positions 126 and 127, respectively, in SEQ ID NO:9;
v. V, Y, and R or conservative substitutions thereof at positions that correspond to positions 149, 150, and 151, respectively, in SEQ ID NO:9;
vi. P, E, I, F, Q, R, S, and V or conservative substitutions thereof at positions that correspond to positions 179, 181, 182, 184, 185, 188, 190, and 191, respectively, in SEQ ID NO:9; and
vii. P, P, P, A, A, P, G, T, A, E, A, P, Q, K, G, and E or conservative substitutions thereof at positions that correspond to positions 220, 222, 229, 230, 231, 234, 248, 258, 260, 262, 264, 172, 175, 279, 283, and 285, respectively, in SEQ ID NO:9,
wherein the polypeptide affects immune signaling, affects intestinal barrier function, affects glucose homeostasis, cholesterol homeostasis, and/or triglyceride homeostasis.

27. The genetically modified host cell of claim 26, the host cell not being of the species Akkermansia muciniphila or Akkermansia glycaniphila.

28. The genetically modified host cell of claim 26, wherein the host cell is of the species Akkermansia muciniphila or Akkermansia glycaniphila.

29. A method for producing a polypeptide, the method comprising the steps of:
(a) culturing a host cell of claim 26 under conditions permitting production of the polypeptide; and
(b) optionally, isolating the polypeptide produced in step (a).

30. A medicament comprising the composition of claim 19.

31. A method of promoting gut mucosal immune system function, for maintaining, restoring or improving glucose and/or cholesterol and/or triglyceride homeostasis, or for maintaining, restoring and/or increasing the physical integrity of the gut mucosal barrier in a mammal, the method comprising:
administering the composition of claim 19 to the mammal, so as to promote gut mucosal immune system function, maintain, restore or improve glucose homeostasis, cholesterol homeostasis, and/or triglyceride homeostasis, or to maintain, restore, and/or increase the gut mucosal barrier's physical integrity.

32. A method of using the composition of claim 19 to prevent and/or treat a subject for a disorder selected from the group consisting of obesity, metabolic syndrome, insulin-deficiency or insulin-resistance related disorders, type 2 diabetes, type 1 diabetes, gestational diabetes, preeclampsia, inflammatory bowel disease (IBD), irritable bowel syndrome (IBS), glucose intolerance, abnormal lipid metabolism, atherosclerosis, hypertension, cardiac pathology, stroke, non-alcoholic fatty liver disease, alcoholic fatty liver disease, hyperglycemia, hepatic steatosis, dyslipidemias, dysfunction of the immune system associated with obesity (weight gain), allergy, asthma, autism, Parkinson's disease, multiple sclerosis, neurodegenerative diseases, depression, other diseases related to compromised barrier function, wound healing, behavioral disorders, alcohol dependence, cardiovascular diseases, high cholesterol, elevated triglycerides, atherosclerosis, sleep apnea, osteoarthritis, gallbladder disease, cancer, and conditions altering the physical integrity of the gut mucosal barrier, the method comprising:
administering the composition to the subject.

33. A method of using the composition of claim 19 to promote anti-inflammatory activity in the gut of a mammal, the method comprising:
administering the composition to the mammal.

34. A method of using the composition of claim 19 to promote weight loss in a mammal, the method comprising:
administering the composition to the mammal.

35. A method for treating a subject to treat and/or preventing a disorder selected from the group consisting of obesity, metabolic syndrome, insulin-deficiency or insulin-resistance related disorders, type 2 diabetes, type 1 diabetes, gestational diabetes, preeclampsia, inflammatory bowel disease (IBD), irritable bowel syndrome (IBS), glucose intolerance, abnormal lipid metabolism, atherosclerosis, hypertension, cardiac pathology, stroke, non-alcoholic fatty liver disease, alcoholic fatty liver disease, hyperglycemia, hepatic steatosis, dyslipidemias, dysfunction of the immune system associated with obesity (weight gain), allergy, asthma, autism, Parkinson's disease, multiple sclerosis, neurodegenerative diseases, depression, other diseases related to compromised barrier function, wound healing, behavioral disorders, alcohol dependence, cardiovascular diseases, high cholesterol, elevated triglycerides, atherosclerosis, sleep apnea, osteoarthritis, gallbladder disease, cancer, and conditions altering the physical integrity of the gut mucosal barrier such as food allergies, immaturity of the gut, the method comprising:
administering to a mammal in need thereof an effective amount of a polypeptide
a) having at least 30% sequence identity with SEQ ID NO:9; and
b) comprising at least one of the following sets of amino acid residues
i. R, S, I, S, A, and P or conservative substitutions thereof at positions that correspond to positions 1, 2, 8, 20, 23, and 27, respectively, in SEQ ID NO:9;
ii. C, K, K, I, and T or conservative substitutions thereof at positions that correspond to positions 92, 93, 95, 97, and 100, respectively, in SEQ ID NO:9;
iii. W, L, G, and F or conservative substitutions thereof at positions that correspond to positions 105, 106, 107, and 108, respectively, in SEQ ID NO:9;
iv. F and E or conservative substitutions thereof at positions that correspond to positions 126 and 127, respectively, in SEQ ID NO:9;
v. V, Y, and R or conservative substitutions thereof at positions that correspond to positions 149, 150, and 151, respectively, in SEQ ID NO:9;
vi. P, E, I, F, Q, R, S, and V or conservative substitutions thereof at positions that correspond to positions 179, 181, 182, 184, 185, 188, 190, and 191, respectively, in SEQ ID NO:9; and
vii. P, P, P, A, A, P, G, T, A, E, A, P, Q, K, G, and E or conservative substitutions thereof at positions that correspond to positions 220, 222, 229, 230, 231, 234, 248, 258, 260, 262, 264, 172, 175, 279, 283, and 285, respectively, in SEQ ID NO:9,
wherein the polypeptide affects immune signaling, affects intestinal barrier function, affects glucose homeostasis, cholesterol homeostasis, and/or triglyceride homeostasis.

36. A method for producing a polypeptide
a) having at least 30% sequence identity with SEQ ID NO:9; and
b) comprising at least one of the following sets of amino acid residues
i. R, S, I, S, A, and P or conservative substitutions thereof at positions that correspond to positions 1, 2, 8, 20, 23, and 27, respectively, in SEQ ID NO:9;
ii. C, K, K, I, and T or conservative substitutions thereof at positions that correspond to positions 92, 93, 95, 97, and 100, respectively, in SEQ ID NO:9;
iii. W, L, G, and F or conservative substitutions thereof at positions that correspond to positions 105, 106, 107, and 108, respectively, in SEQ ID NO:9;
iv. F and E or conservative substitutions thereof at positions that correspond to positions 126 and 127, respectively, in SEQ ID NO:9;
v. V, Y, and R or conservative substitutions thereof at positions that correspond to positions 149, 150, and 151, respectively, in SEQ ID NO:9;
vi. P, E, I, F, Q, R, S, and V or conservative substitutions thereof at positions that correspond to positions 179, 181, 182, 184, 185, 188, 190, and 191, respectively, in SEQ ID NO:9;
vii. P, P, P, A, A, P, G, T, A, E, A, P, Q, K, G, and E or conservative substitutions thereof at positions that correspond to positions 220, 222, 229, 230, 231, 234, 248, 258, 260, 262, 264, 172, 175, 279, 283, and 285, respectively, in SEQ ID NO:9,
wherein the polypeptide affects immune signaling, affects intestinal barrier function, affects glucose homeostasis, cholesterol homeostasis, and/or triglyceride homeostasis, the method comprising the steps of:
culturing bacteria of the species Akkermansia muciniphila or Akkermansia glycaniphila in a suitable culture medium; and
optionally, isolating the polypeptide produced in step (a).

37. A method of promoting gut mucosal immune system function, for maintaining, restoring or improving glucose and/or cholesterol and/or triglyceride homeostasis, or for maintaining, restoring and/or increasing the physical integrity of the gut mucosal barrier in a mammal, the method comprising:
administering the host cell of claim 26 to the mammal so as to promote gut mucosal immune system function, or maintain, restore or improve glucose homeostasis, cholesterol homeostasis, and/or triglyceride homeostasis, or to maintain, restore, and/or increase the gut mucosal barrier's physical integrity.

38. A method of using the host cell of claim 26, to treat a subject so as to prevent and/or treat a disorder selected from the group consisting of obesity, metabolic syndrome, insulin-deficiency or insulin-resistance related disorders, type 2 diabetes, type 1 diabetes, gestational diabetes, preeclampsia, inflammatory bowel disease (IBD), irritable bowel syndrome (IBS), glucose intolerance, abnormal lipid metabolism, atherosclerosis, hypertension, cardiac pathology, stroke, non-alcoholic fatty liver disease, alcoholic fatty liver disease, hyperglycemia, hepatic steatosis, dyslipidemias, dysfunction of the immune system associated with obesity (weight gain), allergy, asthma, autism, Parkinson's disease, multiple sclerosis, neurodegenerative diseases, depression, other diseases related to compromised barrier function, wound healing, behavioral disorders, alcohol dependence, cardiovascular diseases, high cholesterol, elevated triglycerides, atherosclerosis, sleep apnea, osteoarthritis, gallbladder disease, cancer, and conditions altering the physical integrity of the gut mucosal barrier, the method comprising:
administering the host cell to the subject.

39. A method of using the host cell of claim 26 to promote anti-inflammatory activity in the gut of a mammal, the method comprising:
administering the host cell to the mammal.

40. A method of using the host cell of claim 26 to promote weight loss in a mammal, the method comprising:
administering the host cell to the mammal.


SEQUENCE LISTING (20 sequences)

SEQ ID NO: 1 - protein, 287 aa, Akkermansia muciniphila
IVNSKRSELDKKISIAAKEIKSANAAEITPSRSSNEELEKELNRYAKAVGSLETAYKPFLASSALVPTTPTAFQNELKTFRDSLISSCKKKNILITDTSSWLGFQVYSTQAPSVQAASTLGFELKAINSLVNKLAECGLSKFIKVYRPQLPIETPANNPEESDEADQAPWTPMPLEIAFQGDRESVLKAMNAITGMQDYLFTVNSIRIRNERMMPPPIANPAAAKPAAAQPATGAASLTPADEAAAPAAPAIQQVIKPYMGKEQVFVQVSLNLVHFNQPKAQEPSED

SEQ ID NO: 2 - DNA, 864 bp, Akkermansia muciniphila
atcgtcaattccaaacgcagtgaactggacaaaaaaatcagcatcgccgccaaggaaatcaagtccgccaatgctgcggaaatcactccgagccgatcatccaacgaagagctggaaaaagaactgaaccgctatgccaaggccgtgggcagcctggaaacggcctacaagcccttccttgcctcctccgcgctggtccccaccacgcccacggcattccagaatgaactgaaaacattcagggattccctgatctcctcctgcaagaaaaagaacattctcataacggacacatcctcctggctcggtttccaggtttacagcacccaggctccctctgttcaggcggcctccacgctgggttttgaattgaaagccatcaacagcctggtcaacaaactggcggaatgcggcctgtccaaattcatcaaggtgtaccgcccccagctccccattgaaaccccggcgaacaatccggaagaatcggacgaagccgaccaggccccatggactcccatgcctctggaaatagccttccagggcgaccgggaaagtgtattgaaagccatgaacgccataaccggcatgcaggactatctgttcacggtcaactccatccgtatccgcaacgaacggatgatgccccctcccatcgccaatccggcagccgccaaacctgccgcggcccaacccgccacgggtgcggcttccctgactccggcggatgaggcggctgcacctgcagccccggccatccagcaagtcatcaagccttacatgggcaaggagcaggtctttgtccaggtctccctgaatctggtccacttcaaccagcccaaggctcaggaaccgtctgaagattaa

SEQ ID NO: 3 - protein, 30 aa, Akkermansia muciniphila
MSNWITDNKPAAMVAGVGLLLFLGLSATGY

SEQ ID NO: 4 - DNA, 90 bp, Akkermansia muciniphila
atgagcaattggattacagacaacaagcccgccgccatggtcgcgggcgtgggacttctcttattcctggggttatccgcgacagggtac

SEQ ID NO: 5 - protein, 287 aa, Akkermansia muciniphila
IVNSKRSELDKKISIAAKEIKSANAAEITPCRSSNEDLEKELNRYAKAVNSLETAYKPFLASSALVPTTPTAFQNELKTFRDSLISSCKKKNILITDTSNWLGFQVYSTQAPSVQAASTLGFELKAINSLVNKLTECGLSKFIKVYRPQLPIETPANNPEESDEADQSPWTPMPLEIAFQGDRESVLNAINAITGMQDYLFTINSIRIRNERMMPPPIANPAAAKPAADQPATGAASLTPADEAAAPAAPAIQQVIKPYMGKEQIFVQVSLNLIHFNQPKAQEPSED

SEQ ID NO: 6 - protein, 288 aa, Akkermansia muciniphila
IVNSKRSELDKKISVASKEIKSANAAEITPSRASNEELEKELNRYAKAVTSLETAYKPFLASSALVPTTPTAFQNELKTFRDALIASCKKKNILITDTSSWLGFQVYSTQAPSVQAASTLGFELKAVNSLVNKLTDCGLSKFIKVYRPQLPIENPANNPEEDADEPNQAPWTPMPLEIAFQGNRESVLKAMNAITDSQDYLFTVNSIRIRNERMMPPPIANPAAAKPAAAQPAAGAASLTPADEAAAPAAPAIQQLIKPYMGKEQIFVQVSLNLVHFNQPKAQEPSED

SEQ ID NO: 7 - protein, 289 aa, Akkermansia muciniphila
MVNSKRSELDKKISVASKEIKSANAAEITPSRTSNNELEKELNRYAKAVTNLETAYKPFLASSALVPTTPTAFQNELKTFRDALIAACKKKNIQITDTSSWLGFQVYSTQAPSVQAASTLGFELKAVNSLANKLTDCGLTKFIKVYRPQLPIENPANNPEEEAEEPNQAPWSPMPLEIAFQGDRESVLKAMNAITDSQDYLFTVNSIRIRNERMMPPPIAGPAAPKPAAAQSAAGAADLRPADEAAAQSAAPAIQQVIKPYMGKEQIFVQVSLNLVHFNQPKAQEPSED

SEQ ID NO: 8 - protein, 288 aa, Akkermansia sp. KLE1797
MANSERSDLDKKIKSASQEIKSANAAAITPSHTSNKELEKELNRYAKAIGNLETAYKPFMASSVLAPTTPTAFQNELKAFRESLIASCKEKNIQITDTSSWLGFQLYSTQAPSVQATPTLTFEMKAINSLVNKLTDCGLTKFIKVYRSQLPIENPARNTEDEEDSDQKAPWTGMPLEIAFQGDRGSVLKAMNAITDSQEYLFTVNSIRIRNERMMPPPITNPAAAQPASAQPQTGAASLTPAGEAAAPAEPPIQQIIKPYMGKEQVMVQVSLNLVHFAQPKAQEPSED

SEQ ID NO: 9 - protein, 301 aa, Akkermansia glycaniphila
RSASQDNIASIEEGQSTLDSDRAKRFPSNEQSLPEVNAAATRAAAIKEQILASTASFGQTVETATTVDGRPINGKELQDKLNTLHNKLEQLCKEKDIKLTPEASWLGFSAFRSVTPNESDAPDLSFELSGIDHFVNTVAANGAVSITKVYRPTVSEPADKTGKPKPAAKKNTGDWNTLPFEISFQAKRGSVGSILESIAQDKEYCYYITGMRIASDLTTPVPLDPFKKPAAPQPEETATAVSDIIDDGLGGGDPLGGTPAAEPAPAPEEVRPAAQTVAKQILGNETIRVYIACELVRFNTP

SEQ ID NO: 10 - DNA, 103 bp, Artificial Sequence
  Synthetic oligonucleotide
taatacgactcactataggggcccaagttcacttaaaaaggagatcaacaatgaaagcaattttcgtactgaaacatcttaatcatgcaggggagggtttcta

SEQ ID NO: 11 - DNA, 45 bp, Artificial Sequence
  Synthetic oligonucleotide
atgcatcatcatcatcatcatcatgaaaacctgtacttccaatcc

SEQ ID NO: 12 - DNA, 53 bp, Artificial Sequence
  Synthetic oligonucleotide
tgccgactcagttgctgcttctactgggcgccccgcttcggcggggttttttt

SEQ ID NO: 13 - DNA, 18 bp, Artificial Sequence
  Synthetic oligonucleotide
tgccgactcagttgctgc

SEQ ID NO: 14 - DNA, 27 bp, Artificial Sequence
  Synthetic oligonucleotide
ggattggaagtacaggttttcatgatg

SEQ ID NO: 15 - DNA, 864 bp, Akkermansia municiphila
atcgtcaatagtaaacgctctgagctggataagaagatctctatcgccgctaaggagatcaagtcggctaatgccgccgagatcaccccctgtcgctctagtaatgaggatcttgagaaagagctgaatcgctatgccaaggcggtcaatagtctggagaccgcctataaaccttttctggcctcgagtgcccttgttcccaccacccctaccgcctttcagaatgaactgaaaacattcagagacagcctaataagcagctgcaaaaaaaaaaacatactaataacagacacaagcaactggctaggattccaagtatacagcacacaagcaccaagcgtacaagcagcaagcacactaggattcgaactaaaagcaataaacagcctagtaaacaaactaacagaatgcggactaagcaaattcataaaagtatacagaccacaactaccaatagaaacaccagcaaacaacccagaagaaagcgacgaagcagaccaaagcccatggacaccaatgccactagaaatagcattccaaggagacagagaaagcgtactaaacgcaataaacgcaataacaggaatgcaagactacctattcacaataaacagcataagaataagaaacgaaagaatgatgccaccaccaatagcaaacccagcagcagcaaaaccagcagcagaccaaccagcaacaggagcagcaagcctaacaccagcagacgaagcagcagcaccagcagcaccagcaatacaacaagtaataaaaccatacatgggaaaagaacaaatattcgtacaagtaagcctaaacctaatacacttcaaccaaccaaaagcacaagaaccaagcgaagactaa

SEQ ID NO: 16 - DNA, 867 bp, Akkermansia municiphila
atcgtcaattcgaagcgctcagagctggataagaagatctccgttgccagtaaagagatcaagagtgccaatgccgctgagatcaccccctcgcgcgcctctaatgaggaactggagaaagaacttaatcgctatgccaaagccgttaccagtctggagaccgcctataagccctttctggcctcttctgccctggtccccaccactcctaccgcctttcagaatgagctgaaaacattcagagacgcactaatagcaagctgcaaaaaaaaaaacatactaataacagacacaagcagctggctaggattccaagtatacagcacacaagcaccaagcgtacaagcagcaagcacactaggattcgaactaaaagcagtaaacagcctagtaaacaaactaacagactgcggactaagcaaattcataaaagtatacagaccacaactaccaatagaaaacccagcaaacaacccagaagaagacgcagacgaaccaaaccaagcaccatggacaccaatgccactagaaatagcattccaaggaaacagagaaagcgtactaaaagcaatgaacgcaataacagacagccaagactacctattcacagtaaacagcataagaataagaaacgaaagaatgatgccaccaccaatagcaaacccagcagcagcaaaaccagcagcagcacaaccagcagcaggagcagcaagcctaacaccagcagacgaagcagcagcaccagcagcaccagcaatacaacaactaataaaaccatacatgggaaaagaacaaatattcgtacaagtaagcctaaacctagtacacttcaaccaaccaaaagcacaagaaccaagcgaagactaa

SEQ ID NO: 17 - DNA, 870 bp, Akkermansia municiphila
atggtcaattcgaaacgcagtgagctggataaaaagatctccgtcgcctctaaagagatcaagagtgccaatgccgccgagatcacccctagtcgcacctctaataatgagctggagaaagagctgaatcgctatgccaaggccgtgaccaatctggaaaccgcctataagccctttctggcctcttcggccctggttcctaccacccccaccgcctttcagaatgagctgaaaacattcagagacgcactaatagcagcatgcaaaaaaaaaaacatacaaataacagacacaagcagctggctaggattccaagtatacagcacacaagcaccaagcgtacaagcagcaagcacactaggattcgaactaaaagcagtaaacagcctagcaaacaaactaacagactgcggactaacaaaattcataaaagtatacagaccacaactaccaatagaaaacccagcaaacaacccagaagaagaagcagaagaaccaaaccaagcaccatggagcccaatgccactagaaatagcattccaaggagacagagaaagcgtactaaaagcaatgaacgcaataacagacagccaagactacctattcacagtaaacagcataagaataagaaacgaaagaatgatgccaccaccaatagcaggaccagcagcaccaaaaccagcagcagcacaaagcgcagcaggagcagcagacctaagaccagcagacgaagcagcagcacaaagcgcagcaccagcaatacaacaagtaataaaaccatacatgggaaaagaacaaatattcgtacaagtaagcctaaacctagtacacttcaaccaaccaaaagcacaagaaccaagcgaagactaa

SEQ ID NO: 18 - DNA, 867 bp, Akkermansia sp. KLE1797
atggctaattcggagcgcagtgatctggataagaaaatcaagtctgctagtcaggagatcaagagtgccaatgccgccgccatcaccccctcgcatacctcgaataaagagctggagaaagagctgaatcgctatgctaaggctatcggcaatcttgagaccgcttataagccctttatggcctcttcggttctggctcccaccacccctaccgcctttcagaatgagctgaaagcattcagagaaagcctaatagcaagctgcaaagaaaaaaacatacaaataacagacacaagcagctggctaggattccaactatacagcacacaagcaccaagcgtacaagcaacaccaacactaacattcgaaatgaaagcaataaacagcctagtaaacaaactaacagactgcggactaacaaaattcataaaagtatacagaagccaactaccaatagaaaacccagcaagaaacacagaagacgaagaagacagcgaccaaaaagcaccatggacaggaatgccactagaaatagcattccaaggagacagaggaagcgtactaaaagcaatgaacgcaataacagacagccaagaatacctattcacagtaaacagcataagaataagaaacgaaagaatgatgccaccaccaataacaaacccagcagcagcacaaccagcaagcgcacaaccacaaacaggagcagcaagcctaacaccagcaggagaagcagcagcaccagcagaaccaccaatacaacaaataataaaaccatacatgggaaaagaacaagtaatggtacaagtaagcctaaacctagtacacttcgcacaaccaaaagcacaagaaccaagcgaagactaa

SEQ ID NO: 19 - DNA, 867 bp, Akkermansia glycaniphila; identical to SEQ ID NO: 18

SEQ ID NO: 20 - DNA, 927 bp, Akkermansia glycaniphila
cgtagtgctagtcaggataatatcgcctcgatcgaggagggccagtctaccctggatagtgatcgcgccaagcgctttccctcgaatgagcagtcgctgcccgaggtcaatgccgccgctacccgcgctgccgccatcaaggagcagatccttgccagtaccgcttcctttggccagaccgtcgagaccgccaccaccgtcgatggccggcccatcaatggcaaggagctgcaggataagctgaataccctgcacaacaaactagaacaactatgcaaagaaaaagacataaaactaacaccagaagcaagctggctaggattcagcgcattcagaagcgtaacaccaaacgaaagcgacgcaccagacctaagcttcgaactaagcggaatagaccacttcgtaaacacagtagcagcaaacggagcagtaagcataacaaaagtatacagaccaacagtaagcgaaccagcagacaaaacaggaaaaccaaaaccagcagcaaaaaaaaacacaggagactggaacacactaccattcgaaataagcttccaagcaaaaagaggaagcgtaggaagcatactagaaagcatagcacaagacaaagaatactgctactacataacaggaatgagaatagcaagcgacctaacaacaccagtaccactagacccattcaaaaaaccagcagcaccacaaccagaagaaacagcaacagcagtaagcgacataatagacgacggactaggaggaggagacccactaggaggaacaccagcagcagaaccagcaccagcaccagaagaagtaagaccagcagcacaaacagtagcaaaacaaatactaggaaacgaaacaataagagtatacatagcatgcgaactagtaagattcaacacaccagtaaaaaaacaaagcaaaaaataa